SEQUENCE LISTING (12 sequences)

SEQ ID NO: 1 - protein, 129 aa, Mus musculus
MRPRGAPLLAVVLVLGLHALVEGEKPSPCRCSRLTPHNRKNCGFPGITSEQCFDLGCCFDSSVAGVPWCFHPLPNQESEQCVMEVSARKNCGYPGISPEDCASRNCCFSNLIFEVPWCFFPQSVEDCHY

SEQ ID NO: 2 - protein, 129 aa, Homo sapiens
MGRRDAQLLAALLVLGLCALAGSEKPSPCQCSRLSPHNRTNCGFPGITSDQCFDNGCCFDSSVTGVPWCFHPLPKQESDQCVMEVSDRRNCGYPGISPEECASRKCCFSNFIFEVPWCFFPKSVEDCHY

SEQ ID NO: 3 - protein, 106 aa, Mus musculus
EKPSPCRCSRLTPHNRKNCGFPGITSEQCFDLGCCFDSSVAGVPWCFHPLPNQESEQCVMEVSARKNCGYPGISPEDCASRNCCFSNLIFEVPWCFFPQSVEDCHY

SEQ ID NO: 4 - protein, 106 aa, Homo sapiens
EKPSPCQCSRLSPHNRTNCGFPGITSDQCFDNGCCFDSSVTGVPWCFHPLPKQESDQCVMEVSDRRNCGYPGISPEECASRKCCFSNFIFEVPWCFFPKSVEDCHY

SEQ ID NO: 5 - protein, 103 aa, Mus musculus
SPCRCSRLTPHNRKNCGFPGITSEQCFDLGCCFDSSVAGVPWCFHPLPNQESEQCVMEVSARKNCGYPGISPEDCASRNCCFSNLIFEVPWCFFPQSVEDCHY

SEQ ID NO: 6 - protein, 103 aa, Homo sapiens
SPCQCSRLSPHNRTNCGFPGITSDQCFDNGCCFDSSVTGVPWCFHPLPKQESDQCVMEVSDRRNCGYPGISPEECASRKCCFSNFIFEVPWCFFPKSVEDCHY

SEQ ID NO: 7 - protein, 45 aa, Mus musculus
CRCSRLTPHNRKNCGFPGITSEQCFDLGCCFDSSVAGVPWCFHPL

SEQ ID NO: 8 - protein, 45 aa, Homo sapiens
CQCSRLSPHNRTNCGFPGITSDQCFDNGCCFDSSVTGVPWCFHPL

SEQ ID NO: 9 - protein, 44 aa, Mus musculus
EQCVMEVSARKNCGYPGISPEDCASRNCCFSNLIFEVPWCFFPQ

SEQ ID NO: 10 - protein, 44 aa, Homo sapiens
DQCVMEVSDRRNCGYPGISPEECASRKCCFSNFIFEVPWCFFPK

SEQ ID NO: 11 - protein, 16 aa, Mus musculus
EVPWCFFPQSVEDCHY

SEQ ID NO: 12 - protein, 16 aa, Homo sapiens
EVPWCFFPKSVEDCHY

(19) United States
(12) Patent Application Publication
(10) Pub. No.: US 2021/0301013 A1
(43) Pub. Date: Sep. 30, 2021

(54) Methods and Compositions for Treating Aging-Associated Impairments with Trefoil Factor Family Member 2 Modulators

(71) Applicant: Alkahest, Inc., San Carlos, CA (US)

(72) Inventors: Eva Czirr, Foster City, CA (US); Onkar S. Dhande, San Francisco, CA (US); S. Sakura Minami, San Francisco, CA (US); Balazs Szoke, San Carlos, CA (US); Cindy Fu-Jeng Yang, San Francisco, CA (US)

(21) Appl. No.: 17/318,875

(22) Filed: May 12, 2021

Related U.S. Application Data
(63) Continuation-in-part of application No. 17/104,344, filed on Nov. 25, 2020.
(60) Provisional application No. 62/940,477, filed on Nov. 26, 2019.
(60) Provisional application No. 63/071,515, filed on Aug. 28, 2020.

(51) Int. Cl. C07K 16/26; A61P 25/28

(52) U.S. Cl. CPC C07K 16/26; C07K 2317/20; C07K 2317/76; A61P 25/28

(57) ABSTRACT

Methods and compositions for treating and/or preventing aging-related conditions are described. The compositions used in the methods include agents modulating the biological concentrations of trefoil factor family member 2 (TFF2) with efficacy in treating and/or preventing aging-related conditions such as neurocognitive disorders.

### 1. CROSS REFERENCE TO RELATED APPLICATIONS

This application is a continuation-in-part of U.S. patent application Ser. No. 17/104,344 filed Nov. 25, 2020, which application, pursuant to 35 U.S.C. § 119 (e), claims priority to the filing date of U.S. Provisional Patent Application No. 62/940,477, filed Nov. 26, 2019; and U.S. Provisional Patent Application No. 63/071,515, filed Aug. 28, 2020; the disclosures of which applications are herein incorporated by reference.

### 2. FIELD OF THE INVENTION

This invention pertains to the prevention and treatment of aging-related conditions. The invention relates to the use of agents modulating the biological concentrations of trefoil factor family member 2 (TFF2) with efficacy in treating and/or preventing aging-related conditions such as neurocognitive and neurodegenerative disorders.

### 3. SUMMARY

Aging in an organism is accompanied by an accumulation of changes over time. In the nervous system, aging is accompanied by structural and neurophysiological changes that drive cognitive decline and susceptibility to degenerative disorders in healthy individuals. (Heeden & Gabrieli, “Insights into the ageing mind: a view from cognitive neuroscience,” Nat. Rev. Neurosci. (2004) 5: 87-96; Raz et al., “Neuroanatomical correlates of cognitive aging: evidence from structural magnetic resonance imaging,” Neuropsychology (1998) 12:95-114; Mattson & Magnus, “Ageing and neuronal vulnerability,” Nat. Rev. Neurosci. (2006) 7: 278-294; and Rapp & Heindel, “Memory systems in normal and pathological aging,” Curr. Opin. Neurol. (1994) 7:294-298). Included in these changes are synapse loss and the loss of neuronal function that results. Thus, although significant neuronal death is typically not observed during the natural aging process, neurons in the aging brain are vulnerable to sub-lethal age-related alterations in structure, synaptic integrity, and molecular processing at the synapse, all of which impair cognitive function.

In addition to the normal synapse loss during natural aging, synapse loss is an early pathological event common to many neurodegenerative conditions and is the best correlate to the neuronal and cognitive impairment associated with these conditions. Indeed, aging remains the single most dominant risk factor for dementia-related neurodegenerative diseases such as Alzheimer's disease (AD) (Bishop et al., “Neural mechanisms of ageing and cognitive decline,” Nature (2010) 464: 529-535 (2010); Heeden & Gabrieli, “Insights into the ageing mind: a view from cognitive neuroscience,” Nat. Rev. Neurosci. (2004) 5:87-96; Mattson & Magnus, “Ageing and neuronal vulnerability,” Nat. Rev. Neurosci. (2006) 7:278-294).

As human lifespan increases, a greater fraction of the population suffers from aging associated cognitive impairments, making it crucial to elucidate means by which to maintain cognitive integrity by protecting against, or even counteracting, the effects of aging (Hebert et al., “Alzheimer disease in the US population: prevalence estimates using the 2000 census,” Arch. Neurol. (2003) 60:1119-1122; Bishop et al., “Neural mechanisms of ageing and cognitive decline,” Nature (2010) 464:529-535).

Trefoil factor family member 2 (TFF2, also known as spasmolytic polypeptide) is a small peptide member of the trefoil family of peptides. The trefoil family of peptides are small (7-12 kDa) protease-resistant proteins secreted by the gastrointestinal mucosa. TFF2 is predominantly found in the epithelium of the gut, but also found in immune cells, lymphoid tissues, the central nervous system, specifically the hypothalamus, and the endocrine system, specifically the anterior pituitary. In its primary area of expression, the gastric epithelium and duodenal Brunner's glands, it is usually expressed with the mucin MUC6, and together they work in the formation and stabilization of the mucus barrier. TFF2 is also present in the human gastric juice at concentrations between 1 and 20 μg/ml (May, et al., “The human two domain trefoil protein, TFF2, is glycosylated in vivo in the stomach,” Gut (2000) 46:454-459).

Mammalian TFF2 contains two trefoil or P domains, unlike the other mammalian trefoil peptides. These domains contain multiple secondary structural elements, which suggests multiple pharmacophores and matches with the multiple observed functions of TFF. However, little is currently known about the molecular mechanisms of TFF2, and all attempts have so far failed to convincingly demonstrate a typical transmembrane receptor. TFF2 has also been reported to activate PAR4, which likely contributes to mucosal healing (Zhang Y, et al., “Activation of protease-activated receptor (PAR) 1 by frog trefoil factor (TFF) 2 and PAR4 by human TFF2,” Cell Mol Life Sci. (2011) 68:3771-3780). Porcine TFF2 binds non-covalently to integrin β1, which plays an important role in cell migration that is enhanced by TFF peptides (Hoffmann W., “TFF2, a MUC6-binding lectin stabilizing the gastric mucus barrier and more,” Int J Oncol. (2015) 47:806-816; Otto W, Thim L., “Trefoil factor family-interacting proteins,” Cell Mol Life Sci. (2005) 62:2939-2946). Porcine TFF2 has also been found to bind non-covalently to the cysteine-rich repetitive glycoprotein (MW>340 kDa) DMBT1 (formerly: hensin, muclin), an extracellular matrix-associated multifunctional protein playing a role in mucosal innate immunity and protection (Hoffmann W., “TFF2, a MUC6-binding lectin stabilizing the gastric mucus barrier and more,” Int J Oncol. (2015) 47:806-816; Albert T K, et al., “Human intestinal TFF3 forms disulfide-linked heteromers with the mucus-associated FCGBP protein and is released by hydrogen sulfide,” J Proteome Res. (2010) 9:3108-3117). Intravenously administered TFF2 has been found to have been taken up by mucous neck cells, parietal cells, and pyloric gland cells and subsequently appeared in the mucus layer, which could be an indication for receptor-mediated transcytosis (Poulsen S S, Thulesen J, Nexø E and Thim L, “Distribution and metabolism of intravenously administered trefoil factor 2/porcine spasmolytic polypeptide in the rat,” Gut (1998) 43:240-247).

TFF2 is an important part of the viscous gastric mucus barrier, which has multiple physiological functions. The mucus barrier is a biofilm that lubricates the passage of undigested food and protects the epithelium from mechanical damage and pepsin digestion. It is essential for maintaining a pH gradient towards the acidic gastric juice, and it supports and also restricts the adhesion and colonization of microorganisms (such as H. pylori) (Allen A, “Gastrointestinal mucus. Section 6: The gastrointestinal System,” In: Handbook of physiology, Vol. III, Schultz S G (ed.) Am Physiol Soc., Bethesda, Md. (1989) pp. 359-382). TFF2 can be considered a lectin, stabilizing the gastric mucus barrier and thereby affecting its viscoelastic properties (Sturmer R, et al., “Commercial porcine gastric mucin preparations, also used as artificial saliva, are a rich source for the lectin TFF2: in vitro binding studies,” Chembiochem. (2018) 19:2598-2608; Hanisch F G, et al., “Human trefoil factor 2 is a lectin that binds alpha-GlcNAc-capped mucin glycans with antibiotic activity against Helicobacter pylori,” J Biol Chem. (2014) 289:27363-27375). TFF2 binds highly specifically to the GlcNAcα1→4Galβ1→R moiety of MUC6, and the terminal α-GlcNAc has antimicrobial activity against Helicobacter pylori, which might also adhere to the LacdiNAc oligosaccharide of TFF2 via LabA, suggesting a colonization mechanism (Hoffmann W., “TFF2, a MUC6-binding lectin stabilizing the gastric mucus barrier and more,” Int J Oncol. (2015) 47:806-816; Sturmer R, et al., “Commercial porcine gastric mucin preparations, also used as artificial saliva, are a rich source for the lectin TFF2: in vitro binding studies,” Chembiochem. (2018) 19:2598-2608; Hanisch F G, et al., “Human trefoil factor 2 is a lectin that binds alpha-GlcNAc-capped mucin glycans with antibiotic activity against Helicobacter pylori,” J Biol Chem. (2014) 289:27363-27375).

In the central nervous system, TFF2 has been found to be expressed and modulated in the hypothalamus in relation to appetite, satiety, and body weight (Giorgio, et al., “Trefoil Factor Family Member 2 (Tff2) KO Mice Are protected from High-Fat Diet-Induced Obesity,” Obesity (2013) 21: 1389-1395). TFF2 KO mice were found to store energy less efficiently than WT mice and gained less weight and fat mass than WT mice (Giorgio, et al., “Trefoil Factor Family Member 2 (Tff2) KO Mice Are protected from High-Fat Diet-Induced Obesity,” Obesity (2013) 21: 1389-1395). TFF2 has also been found in the anterior pituitary of the mouse brain, where it likely is released to the rest of the body (Hinz M, Schwegler H, Chwieralski C E, Laube G, Linke R, Pohle W and Hoffmann W, “Trefoil factor family (TFF) expression in the mouse brain and pituitary: Changes in the developing cerebellum,” Peptides (2004) 25: 827-832).

The present invention discloses the relationship between age and relative serum plasma TFF2 levels, where such TFF2 levels increase with age. The invention also discloses methods to treat an adult mammal for an aging-associated condition by reducing, blocking, or decreasing the activity of TFF2 in the adult mammal. In light of a long-felt and unmet need in treating diseases of aging such as cognitive impairment, the compositions and methods of the invention address that need by providing a method of administering an agent to reduce, block, or decrease the activity of TFF2 in a subject diagnosed with a cognitive impairment such as, for example and not limitation, Alzheimer's Disease, Parkinson's Disease, Huntington's Disease, Mild Cognitive Impairment, Dementia, and the like.

### 4. SUMMARY

Methods of treating an adult mammal for an aging-associated condition are provided. Aspects of the methods include reducing the trefoil factor family peptide 2 (TFF2) level or its activity in the mammal in a manner sufficient to treat the mammal for the aging-associated impairment. A variety of aging-associated impairments may be treated by practice of the methods, which impairments include cognitive impairments.

### 5. INCORPORATION BY REFERENCE

All publications and patent applications mentioned in this specification are herein incorporated by reference to the same extent as if each individual publication or patent application was specifically and individually indicated to be incorporated by reference.

### 6. BRIEF DESCRIPTION OF THE FIGURES

FIG. 1 shows a “box and whiskers” depiction of the log 2 relative concentrations of TFF2 in plasma from donors of five different age groups. Plasma from males (50 individuals in each age group) aged 18, 30, 45, 55, and 66-years-old were measured using the SomaScan aptamer-based proteomics assay (SomaLogic, Boulder, Colo.). Healthy plasma levels show a highly significant monotonous increase over this age range (p=1.6e-9, Jonckheere-Terpstra trend test). The line within each box indicates the median value.

FIG. 2 shows the results of a radial arm water maze (RAWM) assay which tests reference and working memory performance by requiring the mice to utilize cues to locate escape platforms. (See, e.g., Penley S C, et al., J Vis Exp., (82):50940 (2013)). Young mice treated with hTFF2 made more errors when navigating the maze compared to vehicle-treated mice.

FIG. 3 depicts the results from a Y-maze behavior test. The Y-maze test determines hippocampal-dependent cognition as measured by preference to enter the novel arm (as opposed to the familiar arm) in a cued Y-maze. The percent entries were calculated by normalizing the number of entries in the novel or familiar arm (the two arms of the “Y” maze) to the total entries in the novel and familiar arms. The Wilcoxon matched-pairs signed rank test was used to assess statistical significance between novel and familiar arms in percent of entries. The results of FIG. 2 demonstrate that administration of human TFF2 (hTFF2) to young mice leads to a trend of fewer entries into the novel arm of the Y-maze, indicating a decline in cognitive performance.

FIG. 4 shows quantitative PCR (qPCR) of hippocampal mRNA from hTFF2-treated and vehicle-treated mice. The figure shows that there is an increase in expression of an inflammatory marker, IL-6, as compared to vehicle treated mice. (* P<0.05, Mann-Whitney U test).

FIG. 5 shows RT-qPCR of hippocampal cDNA from hTFF2- and vehicle-treated mice. The figure shows that there is a trend in increased expression of a marker for reactive astrocytes, Ggta1, as compared to vehicle-treated mice. Reactive astrocytes are strongly induced by the central nervous system during injury and disease. (Liddelow S A, et al., Nature, 541(7638):481-87 (2017).

FIG. 6 reports that TFF2 inhibition with L-pyroglutamic acid improved cognitive performance as aged mice treated with the inhibitor entered the novel arm significantly more than the familiar arm (p<0.002). Additionally, the difference between novel and familiar arm entries was greater than that observed with vehicle. Data is shown as mean±SEM.

FIG. 7 shows results from quantitative analysis of immunostaining in hippocampi of aged mice treated with the TFF2 inhibitor compared to vehicle. Synapse density was measured as number of synapses per μm³. There was a strong trend towards higher synapse density in the CA1 region of the hippocampus in mice treated with TFF2 inhibitor. Data is shown as mean±SEM.

FIG. 8A is a Western blot demonstrating that TFF2 protein is detected in brain lysate from 22-month-old C57B16 mice. FIG. 8B shows that the anti-TFF2 antibody recognizes both mouse and human recombinant TFF2 and that mouse TFF2 (12 kDa) and human TFF2 (14 kDa) can be glycosylated in vivo.

FIG. 9 describes a TFF2 bioassay for ERK1/2 phosphorylation in Jurkat cells.

FIG. 10 shows a Western blot demonstrating that treatment of Jurkat cells with human TFF2 leads to increased ERK1/2 phosphorylation.

FIG. 11 is a Western blot showing that anti-human TFF2 antibodies have neutralizing activity in Jurkat cells against human TFF2.

FIGS. 12A-12B demonstrates that an anti-TFF2 antibody can neutralize mouse TFF2 activity in Jurkat cells. FIG. 12A shows that mouse TFF2 can induce ERK1/2 phosphorylation in Jurkat cells at higher concentrations. FIG. 12B demonstrates that at lower concentrations mouse TFF2 no longer can induce ERK1/2 phosphorylation. Additionally, the figures together show that anti-human TFF2 antibody clone HSPGE16C can inhibit ERK1/2 phosphorylation with treatment of 100 nM TFF2, but not 300 nM.

FIG. 13 shows a Western blot demonstrating that the HSPGE16C anti-hTFF2 antibody can neutralize mouse TFF2 activity in Jurkat cells in a concentration-dependent manner.

FIG. 14 shows a table of commercially available anti-TFF2 antibodies tested for neutralization of TFF2 activity in Jurkat cells, as well as their immunogen information, the species of TFF2 the antibody recognizes or binds to, the host species the host species that the antibody was raised in, their clonality, and their isotype.

FIG. 15A shows representations of the peptide sequences for full length mouse TFF2, which is labelled SEQ ID NO: 01, and Human TFF2, which is labelled SEQ ID NO; 02, as well as the TFF2 antigens or epitopes used to generate antibodies for specific protein domains. Mouse sequences are represented as black rectangles and human sequences as white rectangles with each peptide region aligned with the full length TFF2 proteins. The antigens include amino acids 24-129 of Mouse TFF2 (SEQ ID NO: 03); amino acids 24-129 of Human TFF2 (SEQ ID NO: 04); amino acids 27-129 of Mouse TFF2 (SEQ ID NO: 05); amino acids 27-129 of Human TFF2 (SEQ ID NO: 06); amino acids 29-73 of Mouse TFF2 (SEQ ID NO: 07); amino acids 29-73 of Human TFF2 (SEQ ID NO: 08); amino acids 79-122 of Mouse TFF2 (SEQ ID NO: 09); amino acids 79-122 of Human TFF2 (SEQ ID NO: 10); amino acids 114-129 of Mouse TFF2 (SEQ ID NO: 11); and amino acids 114-129 of Human TFF2 (SEQ ID NO: 12). These antigen peptide fragments were or can be used for custom TFF2 antibody generation.

FIG. 15B shows a multiple sequence alignment of SEQ ID Nos: 01 through 12 described in FIG. 15A. The alignment was performed using CLUSTAL 0 (1.2.4) (available at https://www.uniprot.org/align/).

FIG. 16 shows the normalized relative pERK/GAPDH values from Western Blots demonstrating the treatment of Jurkat cells with thirteen anti-TFF2 antibodies. The figure shows the results for treatment of Jurkat cells with a concentration of 4 μg/ml for each of the thirteen anti-TFF2 antibodies listed in FIG. 14 compared to treatment with a vehicle, TFF2, and a positive control (mouse SDF-1).

FIG. 17 shows relative pERK 1/2 ELISA expression in Jurkat cells after treatment with the Clone #1-2 anti-TFF2 antibody and a neutralizing rabbit polyclonal antibody. The figure shows that the commercially available Clone #1-2 antibody decreases mouse TFF2 activity in Jurkat cells.

FIG. 18A shows that aged mice treated with human anti-TFF2 antibody froze more after foot shock during training.

FIG. 18B shows that a significant increase in freezing in TFF2 antibody treated mice compared to control antibody treated mice can be detected for 1 minute after the first foot shock.

FIG. 19A shows that aged mice treated with human anti-TFF2 antibody retained memory better than mice treated with control antibody as determined by the contextual fear conditioning assay.

FIG. 19B shows that significant improvement in cognition as determined by percent freezing occurs in TFF2 antibody treated mice compared to control antibody treated mice during the final half of the assay.

FIG. 20A shows that aged mice treated with human anti-TFF2 antibody moved less during contextual fear conditioning testing.

FIG. 20B shows that mice treated with TFF2 antibody exhibited less movement than mice treated with control antibody during the final half of the assay. This is another indicator that inhibition of TFF2 resulted in improved cognitive performance.

FIG. 21 shows that in the hippocampi of mice that underwent the contextual fear conditioning assay and treated with human anti-TFF2 antibody as described above, there was a downward trend in expression of the inflammatory marker, IL-1β, compared to control antibody treated mice.

### 7. DETAILED DESCRIPTION

Methods of treating an adult mammal for an aging-associated impairment are provided. Aspects of the methods include reducing levels of or decreasing the activity of the trefoil factor family peptide 2 (TFF2) in the mammal in a manner sufficient to treat the mammal for the aging-associated impairment. A variety of aging-associated impairments may be treated by practice of the methods, which impairments include cognitive impairments.

Before the present methods and compositions are described, it is to be understood that this invention is not limited to a particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g., polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### 8. METHODS

As summarized above, aspects of the invention include methods of treating an aging-associated impairment in an adult mammal. The aging-associated impairment may manifest in a number of different ways, e.g., as aging-associated cognitive impairment and/or physiological impairment, e.g., in the form of damage to central or peripheral organs of the body, such as but not limited to: cell injury, tissue damage, organ dysfunction, aging associated lifespan shortening and carcinogenesis, where specific organs and tissues of interest include, but are not limited to skin, neuron, muscle, pancreas, brain, kidney, lung, stomach, intestine, spleen, heart, adipose tissue, testes, ovary, uterus, liver and bone; in the form of decreased neurogenesis, etc.

In some embodiments, the aging-associated impairment is an aging-associated impairment in cognitive ability in an individual, i.e., an aging-associated cognitive impairment. By cognitive ability, or “cognition,” it is meant the mental processes that include attention and concentration, learning complex tasks and concepts, memory (acquiring, retaining, and retrieving new information in the short and/or long term), information processing (dealing with information gathered by the five senses), visuospatial function (visual perception, depth perception, using mental imagery, copying drawings, constructing objects or shapes), producing and understanding language, verbal fluency (word-finding), solving problems, making decisions, and executive functions (planning and prioritizing). By “cognitive decline”, it is meant a progressive decrease in one or more of these abilities, e.g., a decline in memory, language, thinking, judgment, etc. By “an impairment in cognitive ability” and “cognitive impairment,” it is meant a reduction in cognitive ability relative to a healthy individual, e.g., an age-matched healthy individual, or relative to the ability of the individual at an earlier point in time, e.g., 2 weeks, 1 month, 2 months, 3 months, 6 months, 1 year, 2 years, 5 years, or 10 years or more previously. Aging-associated cognitive impairments include impairments in cognitive ability that are typically associated with aging, including, for example, cognitive impairment associated with the natural aging process, e.g., mild cognitive impairment (M.C.I.); and cognitive impairment associated with an aging associated disorder, that is, a disorder that is seen with increasing frequency with increasing senescence, e.g., a neurodegenerative condition such as Alzheimer's disease, Parkinson's 5 disease, frontotemporal dementia, Huntington's disease, amyotrophic lateral sclerosis, multiple sclerosis, glaucoma, myotonic dystrophy, vascular dementia, and the like.

By “treatment” it is meant that at least an amelioration of one or more symptoms associated with an aging-associated impairment afflicting the adult mammal is achieved, where amelioration is used in a broad sense to refer to at least a reduction in the magnitude of a parameter, e.g., a symptom associated with the impairment being treated. As such, treatment also includes situations where a pathological condition, or at least symptoms associated therewith, are completely inhibited, e.g., prevented from happening, or stopped, e.g., terminated, such that the adult mammal no longer suffers from the impairment, or at least the symptoms that characterize the impairment. In some instances, “treatment”, “treating” and the like refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” may be any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. Treatment may result in a variety of different physical manifestations, e.g., modulation in gene expression, increased neurogenesis, rejuvenation of tissue or organs, etc. Treatment of ongoing disease, where the treatment stabilizes or reduces the undesirable clinical symptoms of the patient, occurs in some embodiments. Such treatment may be performed prior to complete loss of function in the affected tissues. The subject therapy may be administered during the symptomatic stage of the disease, and in some cases after the symptomatic stage of the disease.

In some instances where the aging-associated impairment is aging-associated cognitive decline, treatment by methods of the present disclosure slows, or reduces, the progression of aging-associated cognitive decline. In other words, cognitive abilities in the individual decline more slowly, if at all, following treatment by the disclosed methods than prior to or in the absence of treatment by the disclosed methods. In some instances, treatment by methods of the present disclosure stabilizes the cognitive abilities of an individual. For example, the progression of cognitive decline in an individual suffering from aging-associated cognitive decline is halted following treatment by the disclosed methods. As another example, cognitive decline in an individual, e.g., an individual 40 years old or older, that is projected to suffer from aging-associated cognitive decline, is prevented following treatment by the disclosed methods. In other words, no (further) cognitive impairment is observed. In some instances, treatment by methods of the present disclosure reduces, or reverses, cognitive impairment, e.g., as observed by improving cognitive abilities in an individual suffering from aging-associated cognitive decline. In other words, the cognitive abilities of the individual suffering from aging-associated cognitive decline following treatment by the disclosed methods are better than they were prior to treatment by the disclosed methods, i.e., they improve upon treatment. In some instances, treatment by methods of the present disclosure abrogates cognitive impairment. In other words, the cognitive abilities of the individual suffering from aging-associated cognitive decline are restored, e.g., to their level when the individual was about 40 years old or less, following treatment by the disclosed methods, e.g., as evidenced by improved cognitive abilities in an individual suffering from aging-associated cognitive decline.

In some instances, treatment of an adult mammal in accordance with the methods results in a change in a central organ, e.g., a central nervous system organ, such as the brain, spinal cord, etc., where the change may manifest in a number of different ways, e.g., as described in greater detail below, including but not limited to molecular, structural and/or functional, e.g., in the form of enhanced neurogenesis.

As summarized above, methods described herein are methods of treating an aging associated impairment, e.g., as described above, in an adult mammal. By adult mammal is meant a mammal that has reached maturity, i.e., that is fully developed. As such, adult mammals are not juvenile. Mammalian species that may be treated with the present methods include canines and felines; equines; bovines; ovines; etc., and primates, including humans. The subject methods, compositions, and reagents may also be applied to animal models, including small mammals, e.g., murine, lagomorpha, etc., for example, in experimental investigations. The discussion below will focus on the application of the subject methods, compositions, reagents, devices and kits to humans, but it will be understood by the ordinarily skilled artisan that such descriptions can be readily modified to other mammals of interest based on the knowledge in the art.

The age of the adult mammal may vary, depending on the type of mammal that is being treated. Where the adult mammal is a human, the age of the human is generally 18 years or older. In some instances, the adult mammal is an individual suffering from or at risk of suffering from an aging-associated impairment, such as an aging-associated cognitive impairment, where the adult mammal may be one that has been determined, e.g., in the form of receiving a diagnosis, to be suffering from or at risk of suffering from an aging associated impairment, such as an aging-associated cognitive impairment. The phrase “an individual suffering from or at risk of suffering from an aging-associated cognitive impairment” refers to an individual that is about 50 years old or older, e.g., 60 years old or older, 70 years old or older, 80 years old or older, and sometimes no older than 100 years old, such as 90 years old, i.e., between the ages of about 50 and 100, e.g., 50, 55, 60, 65, 70, 75, 80, 85 or about 90 years old. The individual may suffer from an aging associated condition, e.g., cognitive impairment, associated with the natural aging process, e.g., M.C.I. Alternatively, the individual may be 50 years old or older, e.g., 60 years old or older, 70 years old or older, 80 years old or older, 90 years old or older, and sometimes no older than 100 years old, i.e., between the ages of about 50 and 100, e.g., 50, 55, 60, 65, 70, 75, 80, 85, 90, 95 or about 100 years old, and has not yet begun to show symptoms of an aging associated condition, e.g., cognitive impairment. In yet other embodiments, the individual may be of any age where the individual is suffering from a cognitive impairment due to an aging-associated disease, e.g., Alzheimer's disease, Parkinson's disease, frontotemporal dementia, Huntington's disease, amyotrophic lateral sclerosis, multiple sclerosis, glaucoma, myotonic dystrophy, dementia, and the like. In some instances, the individual is an individual of any age that has been diagnosed with an aging-associated disease that is typically accompanied by cognitive impairment, e.g., Alzheimer's disease, Parkinson's disease, frontotemporal dementia, progressive supranuclear palsy, Huntington's disease, amyotrophic lateral sclerosis, spinal muscular atrophy, multiple sclerosis, multi-system atrophy, glaucoma, ataxias, myotonic dystrophy, dementia, and the like, where the individual has not yet begun to show symptoms of cognitive impairment.

As summarized above, aspects of the methods include reducing levels of or decreasing the activity of the trefoil factor family peptide 2 (TFF2) in the mammal in a manner sufficient to treat the aging impairment in the mammal, e.g., as described above. By reducing the TFF2 level is meant lowering the amount of TFF2 in the mammal, such as the amount of extracellular TFF2 in the mammal. By decreasing the activity of the TFF2 peptide is meant lowering the ability of TFF2 to act through its mechanism of action, for example, its ability to specifically bind to a receptor or such as through providing an agent that interferes with such binding. Decreasing the activity also may mean interfering with the ability of TFF2 to interact with a substrate molecule necessary for TFF2 to produce its detrimental effects on aging or cognition. While the magnitude of the reduction or decreasing may vary, in some instances the magnitude is 2-fold or greater, such as 5-fold or greater, including 10-fold or greater, e.g., 15-fold or greater, 20-fold or greater, 25-fold or greater (as compared to a suitable control), where in some instances the magnitude is such that the amount of detectable free TFF2 in the circulatory system of the individual is 50% or less, such as 25% or less, including 10% or less, e.g., 1% or less, relative to the amount that was detectable prior to intervention according to the invention, and in some instances the amount is undetectable following intervention.

The TFF2 level may be reduced using any convenient protocol. In some instances, the TFF2 level is reduced by removing systemic TFF2 from the adult mammal, e.g., by removing TFF2 from the circulatory system of the adult mammal. In such instances, any convenient protocol for removing circulatory TFF2 may be employed. For example, blood may be obtained from the adult mammal and extra-corporeally processed to remove TFF2 from the blood to produce TFF2 depleted blood, which resultant TFF2 depleted blood may then be returned to the adult mammal. Such protocols may employ a variety of different techniques in order to remove TFF2 from the obtained blood. For example, the obtained blood may be contacted with a filtering component, e.g., a membrane, etc., which allows passage of TFF2 but inhibits passage of other blood components, e.g., cells, etc. In some instances, the obtained blood may be contacted with a TFF2 absorptive component, e.g., porous bead or particulate composition, which absorbs TFF2 from the blood. In some instances, the obtained blood may be contacted with a TTF2-specific antibody which selectively binds to TFF2, reducing its blood levels. In yet other instances, the obtained blood may be contacted with a TFF2 binding member stably associated with a solid support, such that TFF2 binds to the binding member and is thereby immobilized on the solid support, thereby providing for separation of TFF2 from other blood constituents. The protocol employed may or may not be configured to selectively remove TFF2 from the obtained blood, as desired.

In some embodiments, the TFF2 level is reduced by administering to the mammal an effective amount of a TFF2 level reducing agent. As such, in practicing methods according to these embodiments of the invention, an effective amount of the active agent, e.g., TFF2 modulatory agent, is provided to the adult mammal. In embodiments, of interest as TFF2 modulatory agents are specific TFF2 level reducing agents, by which is meant agents that selectively reduce TFF2 levels to a greater extent than other factors. By way of example and not limitation, such other factors can be other secreted factors such as epidermal growth factor, nerve growth factor, fibroblast growth factor, tumor necrosis factor alpha, thrombopoietin, insulin-like growth factor 1, insulin-like growth factor binding protein 3 and erythropoietin, as well as other proteins of the trefoil factor family such as trefoil factor 1 (TFF1) and trefoil factor 3 (TFF3).

Depending on the particular embodiments being practiced, a variety of different types of active agents may be employed. In some instances, the agent modulates expression of the RNA and/or protein from the gene, such that it changes the expression of the RNA or protein from the target gene in some manner. In these instances, the agent may change expression of the RNA or protein in a number of different ways. In certain embodiments, the agent is one that reduces, including inhibits, expression of a TFF2 protein. Inhibition of TFF2 protein expression may be accomplished using any convenient means, including use of an agent that inhibits TFF2 protein expression, such as, but not limited to: RNAi agents, antisense agents, agents that interfere with a transcription factor binding to a promoter sequence of the TFF2 gene, or inactivation of the TFF2 gene, e.g., through recombinant techniques, etc.

For example, the transcription level of a TFF2 protein can be regulated by gene silencing using RNAi agents, e.g., double-strand RNA (see e.g., Sharp, Genes and Development (1999) 13: 139-141). RNAi, such as double-stranded RNA interference (dsRNAi) or small interfering RNA (siRNA), has been extensively documented in the nematode C. elegans (Fire, et al, Nature (1998) 391:806-811) and routinely used to “knock down” genes in various systems. RNAi agents may be dsRNA or a transcriptional template of the interfering ribonucleic acid which can be used to produce dsRNA in a cell. In these embodiments, the transcriptional template may be a DNA that encodes the interfering ribonucleic acid. Methods and procedures associated with RNAi are also described in published PCT Application Publication Nos. WO 03/010180 and WO 01/68836, the disclosures of which applications are incorporated herein by reference. dsRNA can be prepared according to any of a number of methods that are known in the art, including in vitro and in vivo methods, as well as by synthetic chemistry approaches. Examples of such methods include, but are not limited to, the methods described by Sadher et al., Biochem. Int. (1987) 14:1015; Bhattacharyya, Nature (1990) 343:484; and U.S. Pat. No. 5,795,715, the disclosures of which are incorporated herein by reference. Single-stranded RNA can also be produced using a combination of enzymatic and organic synthesis or by total organic synthesis. The use of synthetic chemical methods enables one to introduce desired modified nucleotides or nucleotide analogs into the dsRNA. dsRNA can also be prepared in vivo according to a number of established methods (see, e.g., Sambrook, et al. (1989) Molecular Cloning: A Laboratory Manual, 2nd ed.; Transcription and Translation (B. D. Hames, and S. J. Higgins, Eds., 1984); DNA Cloning, volumes I and II (D. N. Glover, Ed., 1985); and Oligonucleotide Synthesis (M. J. Gait, Ed., 1984, each of which is incorporated herein by reference). A number of options can be utilized to deliver the dsRNA into a cell or population of cells such as in a cell culture, tissue, organ or embryo. For instance, RNA can be directly introduced intracellularly. Various physical methods are generally utilized in such instances, such as administration by microinjection (see, e.g., Zernicka-Goetz, et al. Development (1997)124:1133-1137; and Wianny, et al., Chromosoma (1998) 107: 430-439). Other options for cellular delivery include permeabilizing the cell membrane and electroporation in the presence of the dsRNA, liposome-mediated transfection, or transfection using chemicals such as calcium phosphate. A number of established gene therapy techniques can also be utilized to introduce the dsRNA into a cell. By introducing a viral construct within a viral particle, for instance, one can achieve efficient introduction of an expression construct into the cell and transcription of the RNA encoded by the construct. Specific examples of RNAi agents that may be employed to reduce TFF2 expression include but are not limited to commercially-available TFF2 siRNAs (see, e.g., MyBioSource (San Diego, Calif.) which provides a commercially-available human TFF2 siRNA (#MBS8204153); OriGene Technologies (Rockville, Md.) which provides three unique commercially-available 27mer human siRNA or shRNA duplexes targeting TFF2 (Item Nos. SR304798, TL308865, TR308865); and ThermoFisher Scientific provides a commercially-available human TFF2 siRNA (Catalog No. AM16708).)

In some instances, antisense molecules can be used to down-regulate expression of a TFF2 gene in the cell. The anti-sense reagent may be antisense oligodeoxynucleotides (ODN), particularly synthetic ODN having chemical modifications from native nucleic acids, or nucleic acid constructs that express such anti-sense molecules as RNA. The antisense sequence is complementary to the mRNA of the targeted protein and inhibits expression of the targeted protein. Antisense molecules inhibit gene expression through various mechanisms, e.g., by reducing the amount of mRNA available for translation, through activation of RNAse H, or steric hindrance. One or a combination of antisense molecules may be administered, where a combination may include multiple different sequences.

Antisense molecules may be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like. Short oligonucleotides, of from 7 to 8 bases in length, can be strong and selective inhibitors of gene expression (see Wagner et al., Nature Biotechnol. (1996) 14:840-844).

A specific region or regions of the endogenous sense strand mRNA sequence are chosen to be complemented by the antisense sequence. Selection of a specific sequence for the oligonucleotide may use an empirical method, where several candidate sequences are assayed for inhibition of expression of the target gene in an in vitro or animal model. A combination of sequences may also be used, where several regions of the mRNA sequence are selected for antisense complementation.

Antisense oligonucleotides may be chemically synthesized by methods known in the art (see Wagner et al. (1993), supra.). Oligonucleotides may be chemically modified from the native phosphodiester structure, in order to increase their intracellular stability and binding affinity. A number of such modifications have been described in the literature, which alter the chemistry of the backbone, sugars or heterocyclic bases. Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O-5′-S-phosphorothioate, 3′-S-5′-O-phosphorothioate, 3′-CH.sub.2-5′-O-phosphonate and 3′-NH-5′-Ophosphoroamidate. Peptide nucleic acids replace the entire ribose phosphodiester backbone with a peptide linkage. Sugar modifications are also used to enhance stability and affinity. The α-anomer of deoxyribose may be used, where the base is inverted with respect to the natural β-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

As an alternative to anti-sense inhibitors, catalytic nucleic acid compounds, e.g. ribozymes, anti-sense conjugates, etc. may be used to inhibit gene expression. Ribozymes may be synthesized in vitro and administered to the patient, or may be encoded on an expression vector, from which the ribozyme is synthesized in the targeted cell (for example, see International patent application WO 9523225, and Beigelman et al. Nucl. Acids Res. (1995) 23:4434-42). Examples of oligonucleotides with catalytic activity are described in WO 9506764. Conjugates of anti-sense ODN with a metal complex, e.g. terpyridylCu(II), capable of mediating mRNA hydrolysis are described in Bashkin et al. Appl. Biochem. Biotechnol. (1995) 54:43-56.

In another embodiment, the TFF2 gene is inactivated so that it no longer expresses a functional protein. By inactivated is meant that the gene, e.g., coding sequence and/or regulatory elements thereof, is genetically modified so that it no longer expresses a functional TFF2 protein, e.g., at least with respect to TFF2 aging impairment activity. The alteration or mutation may take a number of different forms, e.g., through deletion of one or more nucleotide residues, through exchange of one or more nucleotide residues, and the like. One means of making such alterations in the coding sequence is by homologous recombination. Methods for generating targeted gene modifications through homologous recombination are known in the art, including those described in: U.S. Pat. Nos. 6,074,853; 5,998,209; 5,998,144; 5,948,653; 5,925,544; 5,830,698; 5,780,296; 5,776,744; 5,721,367; 5,614,396; 5,612,205; the disclosures of which are herein incorporated by reference.

Also of interest in certain embodiments are dominant negative mutants of TFF2 proteins, where expression of such mutants in the cell result in a modulation, e.g., decrease, in TFF2 mediated aging impairment. Dominant negative mutants of TFF2 are mutant proteins that exhibit dominant negative TFF2 activity. As used herein, the term “dominant-negative TFF2 activity” or “dominant negative activity” refers to the inhibition, negation, or diminution of certain particular activities of TFF2, and specifically to TFF2 mediated aging impairment. Dominant negative mutations are readily generated for corresponding proteins. These may act by several different mechanisms, including mutations in a substrate-binding domain; mutations in a catalytic domain; mutations in a protein binding domain (e.g., multimer forming, effector, or activating protein binding domains); mutations in cellular localization domain, etc. A mutant polypeptide may interact with wild-type polypeptides (made from the other allele) and form a non-functional multimer. In certain embodiments, the mutant polypeptide will be overproduced. Point mutations are made that have such an effect. In addition, fusion of different polypeptides of various lengths to the terminus of a protein, or deletion of specific domains can yield dominant negative mutants. General strategies are available for making dominant negative mutants (see for example, Herskowitz, Nature (1987) 329:219, and the references cited above). Such techniques are used to create loss of function mutations, which are useful for determining protein function. Methods that are well known to those skilled in the art can be used to construct expression vectors containing coding sequences and appropriate transcriptional and translational control signals for increased expression of an exogenous gene introduced into a cell. These methods include, for example, in vitro recombinant DNA techniques, synthetic techniques, and in vivo genetic recombination. Alternatively, RNA capable of encoding gene product sequences may be chemically synthesized using, for example, synthesizers. See, for example, the techniques described in “Oligonucleotide Synthesis”, 1984, Gait, M. J. ed., IRL Press, Oxford.

In yet other embodiments, the agent is an agent that modulates, e.g., inhibits, TFF2 activity by binding to TFF2 and/or inhibiting binding of TFF2 to a second protein, e.g., interleukin 1β. For example, small molecules that bind to the TFF2 and inhibit its activity are of interest. Naturally occurring or synthetic small molecule compounds of interest include numerous chemical classes, such as organic molecules, e.g., small organic compounds having a molecular weight of more than 50 and less than about 2,500 daltons. Candidate agents comprise functional groups for structural interaction with proteins, particularly hydrogen bonding, and typically include at least an amine, carbonyl, hydroxyl or carboxyl group, preferably at least two of the functional chemical groups. The candidate agents may include cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Such molecules may be identified, among other ways, by employing the screening protocols described below.

In certain embodiments, the administered active agent is a TFF2 specific binding member. The term “specific binding” refers to a direct association between two molecules, due to, for example, covalent, electrostatic, hydrophobic, and ionic and/or hydrogen-bond interactions, including interactions such as salt bridges and water bridges. A specific binding member describes a member of a pair of molecules which have binding specificity for one another. The members of a specific binding pair may be naturally derived or wholly or partially synthetically produced. One member of the pair of molecules has an area on its surface, or a cavity, which specifically binds to and is therefore complementary to a particular spatial and polar organization of the other member of the pair of molecules. Thus, the members of the pair have the property of binding specifically to each other. Examples of pairs of specific binding members are antigen-antibody, biotin-avidin, hormone-hormone receptor, receptor-ligand, enzyme-substrate. Specific binding members of a binding pair exhibit high affinity and binding specificity for binding with the each other. In general, useful TFF2 specific binding members exhibit an affinity (Kd) for a target TFF2, such as human TFF2, that is sufficient to provide for the desired reduction in aging associated impairment TFF2 activity. As used herein, the term “affinity” refers to the equilibrium constant for the reversible binding of two agents; “affinity” can be expressed as a dissociation constant (Kd). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater, or more, than the affinity of an antibody for unrelated amino acid sequences. Affinity of a specific binding member to a target protein can be, for example, from about 100 nanomolar (nM) to about 0.1 nM, from about 100 nM to about 1 picomolar (pM), or from about 100 nM to about 1 femtomolar (fM) or more. The term “binding” refers to a direct association between two molecules, due to, for example, covalent, electrostatic, hydrophobic, and ionic and/or hydrogen-bond interactions, including interactions such as salt bridges and water bridges. In some embodiments, the antibodies bind human TFF2 with nanomolar affinity or picomolar affinity. In some embodiments, the antibodies bind human TFF2 with a Kd of less than about 100 nM, 50 nM, 20 nM, 20 nM, or 1 nM. In an embodiment, affinity is determined by surface plasmon resonance (SPR), e.g. as used by Biacore systems. The affinity of one molecule for another molecule is determined by measuring the binding kinetics of the interaction, e.g. at 25° C.

Examples of TFF2 specific binding members include TFF2 antibodies and binding fragments thereof. Non-limiting examples of such antibodies include antibodies directed against any epitope of TFF2. Examples of said epitopes include, by way of example and not limitation the amino acid sequences of SEQ ID NO: 01, SEQ ID NO: 02, SEQ ID NO: 03, SEQ ID NO: 04, SEQ ID NO: 05, SEQ ID NO: 06, SEQ ID NO: 07, SEQ ID NO: 08, SEQ ID NO: 09, SEQ ID NO: 10, SEQ ID NO: 11, and SEQ ID NO: 12. In some embodiment of the invention, said epitopes have at least about any of 90%, 91%, 92%, 93%, 94%, 95%, 96%, 97%, 98%, 99%, or 100% sequence identity to the amino acid sequences of SEQ ID NO: 01, SEQ ID NO: 02, SEQ ID NO: 03, SEQ ID NO: 04, SEQ ID NO: 05, SEQ ID NO: 06, SEQ ID NO: 07, SEQ ID NO: 08, SEQ ID NO: 09, SEQ ID NO: 10, SEQ ID NO: 11, SEQ ID NO: 12.

Also encompassed are bispecific antibodies, i.e., antibodies in which each of the two binding domains recognizes a different binding epitope. The amino acid sequence of human TFF2 is disclosed in May, F. E. B. & Semple, Jennifer & Newton, J. L. & Westley, B. R., “The human two domain trefoil protein, TFF2, is glycosylated in vivo in the stomach,” Gut. (2000) 46: 454-459.

Antibody specific binding members that may be employed include full antibodies or immunoglobulins of any isotype, as well as fragments of antibodies which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotinavidin specific binding pair), and the like. Also encompassed by the term are Fab′, Fv, F(ab′)2, and or other antibody fragments that retain specific binding to antigen, and monoclonal antibodies. An antibody may be monovalent or bivalent.

“Antibody fragments” comprise a portion of an intact antibody, for example, the antigen binding or variable region of the intact antibody. Examples of antibody fragments include Fab, Fab′, F(ab′)₂, and Fv fragments; diabodies; linear antibodies (Zapata et al., Protein Eng. 8(10): 1057-1062 (1995)); single-chain antibody molecules; and multi-specific antibodies formed from antibody fragments. Papain digestion of antibodies produces two identical antigen-binding fragments, called “Fab” fragments, each with a single antigen binding site, and a residual “Fc” fragment, a designation reflecting the ability to crystallize readily. Pepsin treatment yields an F(ab′)2 fragment that has two antigen combining sites and is still capable of cross-linking antigen.

“Fv” is the minimum antibody fragment which contains a complete antigen recognition and -binding site. This region consists of a dimer of one heavy- and one light chain variable domain in tight, non-covalent association. It is in this configuration that the three CDRS of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The “Fab” fragment also contains the constant domain of the light chain and the first constant domain (CH1) of the heavy chain. Fab fragments differ from Fab′ fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2.

“Single-chain Fv” or “sFv” antibody fragments comprise the VH and VL domains of antibody, wherein these domains are present in a single polypeptide chain. In some embodiments, the Fv polypeptide further comprises a polypeptide linker between the VH and VL domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

Antibodies that may be used in connection with the present disclosure thus can encompass monoclonal antibodies, polyclonal antibodies, bispecific antibodies, Fab antibody fragments, F(ab)2 antibody fragments, Fv antibody fragments (e.g., VH or VL), single chain Fv antibody fragments and dsFv antibody fragments. Furthermore, the antibody molecules may be fully human antibodies, humanized antibodies, or chimeric antibodies. In some embodiments, the antibody molecules are monoclonal, fully human antibodies.

The antibodies that may be used in connection with the present disclosure can include any antibody variable region, mature or unprocessed, linked to any immunoglobulin constant region. If a light chain variable region is linked to a constant region, it can be a kappa chain constant region. If a heavy chain variable region is linked to a constant region, it can be a human gamma 1, gamma 2, gamma 3 or gamma 4 constant region, more preferably, gamma 1, gamma 2 or gamma 4 and even more preferably gamma 1 or gamma 4.

In some embodiments, fully human monoclonal antibodies directed against TFF2 are generated using transgenic mice carrying parts of the human immune system rather than the mouse system.

Methods of creating polyclonal and monoclonal antibodies are well known to those having ordinary skill in the art. (See Leenaars M, et al., ILAR J, 46(3):269-79 (2005) and Lu R-M, et al., J Biomed Sci, 27(1) (2020)). Antigens are prepared in order to raise antibodies to the antigens in animal species such as mice, rabbits, goats, rats, sheep, chicken, hamster, and guinea pig by immunizing said animals with an antigen. Adjuvants can also be administered in order to help provoke an immune response. Immunization can be administered via subcutaneous, intradermal, intramuscular, intraperitoneal, or intravenous means for example. Booster injections may follow, depending on the impact of initial immunization. The antibody response is monitored and exsanguination often by euthanasia and heart puncture follows, with purification of the antibodies. Monoclonal antibodies (MAbs) may be created, for example, by producing a single clone of B cells, which are capable of being immortalized by fusion with myeloma cells. This creates a hybridoma cell line that is able to produce an unlimited quantity of MAbs.

Minor variations in the amino acid sequences of antibodies or immunoglobulin molecules are encompassed by the present invention, providing that the variations in the amino acid sequence maintain at least 75%, e.g., at least 80%, 90%, 95%, or 99% of the sequence. In particular, conservative amino acid replacements are contemplated. Conservative replacements are those that take place within a family of amino acids that are related in their side chains. Whether an amino acid change results in a functional peptide can readily be determined by assaying the specific activity of the polypeptide derivative. Fragments (or analogs) of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Sequence motifs and structural conformations may be used to define structural and functional domains in accordance with the invention.

Specific examples of antibody agents that may be employed to reduce TFF2 expression or activity include, but are not limited to commercially-available antibodies (see, e.g., MyBioSource (San Diego, Calif.) which provides a commercially-available human anti-TFF2 polyclonal antibody (#MBS9125301); LifeSpan Biosciences (Seattle, Wash.) which provides a commercially-available human anti-TFF2 polyclonal antibody (Catalog No. LS-A9840-50); R&D Systems (Minneapolis, Minn.) which provides a commercially-available human anti-TFF2 monoclonal antibody (Catalog No. MAB4077); Biorbyt (Cambridge, UK) which provides a commercially-available human anti-TFF2 (Catalog No. orb197800).; ThermoFisher Scientific which provides a commercially-available human anti-TFF2 monoclonal antibody (Catalog No. 4G7C3); and other Anti-TFF2 human antibodies that have also been described before. (See, e.g., Siu L-S, et al., Peptides, 25(5):855-63 (2004)). Methods of making and designing monoclonal antibodies are commonly known to those having ordinary skill in the art and include for example, Greenfield E A, Antibodies: A Laboratory manual, 2nd ed. (2014) and Kohler G, et al., Continuous cultures of fused cells secreting antibody of predefined specificity, Nature 256:495-97 (1975) which are herein incorporated by reference in their entirety).

In those embodiments where an active agent is administered to the adult mammal, the active agent(s) may be administered to the adult mammal using any convenient administration protocol capable of resulting in the desired activity. Thus, the agent can be incorporated into a variety of formulations, e.g., pharmaceutically acceptable vehicles, for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments (e.g., skin creams), solutions, suppositories, injections, inhalants and aerosols. As such, administration of the agents can be achieved in various ways, including oral, buccal, rectal, parenteral, intraperitoneal, intradermal, transdermal, intracheal, etc., administration.

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The agents can be utilized in aerosol formulation to be administered via inhalation. The compounds of the present invention can be formulated into pressurized acceptable propellants such as dichlorodifluoromethane, propane, nitrogen and the like.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Where the agent is a polypeptide, polynucleotide, analog or mimetic thereof, it may be introduced into tissues or host cells by any number of routes, including viral infection, microinjection, or fusion of vesicles. Jet injection may also be used for intramuscular administration, as described by Furth et al., Anal Biochem. (1992) 205:365-368. The DNA may be coated onto gold microparticles, and delivered intradermally by a particle bombardment device, or “gene gun” as described in the literature (see, for example, Tang et al., Nature (1992) 356:152-154), where gold microprojectiles are coated with the DNA, then bombarded into skin cells. For nucleic acid therapeutic agents, a number of different delivery vehicles find use, including viral and non-viral vector systems, as are known in the art.

Those of skill in the art will readily appreciate that dose levels can vary as a function of the specific compound, the nature of the delivery vehicle, and the like. Preferred dosages for a given compound are readily determinable by those of skill in the art by a variety of means.

In those embodiments where an effective amount of an active agent is administered to the adult mammal, the amount or dosage is effective when administered for a suitable period of time, such as one week or longer, including two weeks or longer, such as 3 weeks or longer, 4 weeks or longer, 8 weeks or longer, etc., so as to evidence a reduction in the impairment, e.g., cognition decline and/or cognitive improvement in the adult mammal. For example, an effective dose is the dose that, when administered for a suitable period of time, such as at least about one week, and maybe about two weeks, or more, up to a period of about 3 weeks, 4 weeks, 8 weeks, or longer, will slow e.g., by about 20% or more, e.g., by 30% or more, by 40% or more, or by 50% or more, in some instances by 60% or more, by 70% or more, by 80% or more, or by 90% or more, e.g., will halt, cognitive decline in a patient suffering from natural aging or an aging-associated disorder. In some instances, an effective amount or dose of active agent will not only slow or halt the progression of the disease condition but will also induce the reversal of the condition, i.e., will cause an improvement in cognitive ability. For example, in some instances, an effective amount is the amount that when administered for a suitable period of time, usually at least about one week, and maybe about two weeks, or more, up to a period of about 3 weeks, 4 weeks, 8 weeks, or longer will improve the cognitive abilities of an individual suffering from an aging associated cognitive impairment by, for example 1.5-fold, 2-fold, 3-fold, 4-fold, 5-fold, in some instances 6-fold, 7-fold, 8-fold, 9-fold, or 10-fold or more relative to cognition prior to administration of the blood product.

Where desired, effectiveness of treatment may be assessed using any convenient protocol. Cognition tests and IQ test for measuring cognitive ability, e.g., attention and concentration, the ability to learn complex tasks and concepts, memory, information processing, visuospatial function, the ability to produce and understanding language, the ability to solve problems and make decisions, and the ability to perform executive functions, are well known in the art, any of which may be used to measure the cognitive ability of the individual before and/or during and after treatment with the subject blood product, e.g., to confirm that an effective amount has been administered. These include, for example, the General Practitioner Assessment of Cognition (GPCOG) test, the Memory Impairment Screen, the Mini Mental State Examination (MMSE), the California Verbal Learning Test, Second Edition, Short Form, for memory, the Delis-Kaplan Executive Functioning System test, the Alzheimer's Disease Assessment Scale (ADAS-Cog), the Psychogeriatric Assessment Scale (PAS) and the like. Progression of functional brain improvements may be detected by brain imaging techniques, such as Magnetic Resonance Imaging (MRI) or Positron Emission Tomography (PET) and the like. A wide range of additional functional assessments may be applied to monitor activities of daily living, executive functions, mobility, etc. In some embodiments, the method comprises the step of measuring cognitive ability, and detecting a decreased rate of cognitive decline, a stabilization of cognitive ability, and/or an increase in cognitive ability after administration of the blood product as compared to the cognitive ability of the individual before the blood product was administered. Such measurements may be made a week or more after administration of the blood product, e.g., 1 week, 2 weeks, 3 weeks, or more, for instance, 4 weeks, 6 weeks, or 8 weeks or more, e.g., 3 months, 4 months, 5 months, or 6 months or more.

Biochemically, by an “effective amount” or “effective dose” of active agent is meant an amount of active agent that will inhibit, antagonize, decrease, reduce, or suppress by about 20% or more, e.g., by 30% or more, by 40% or more, or by 50% or more, in some instances by 60% or more, by 70% or more, by 80% or more, or by 90% or more, in some cases by about 100%, i.e., to negligible amounts, and in some instances reverse, the reduction in synaptic plasticity and loss of synapses that occurs during the natural aging process or during the progression of an aging-associated disorder. In other words, cells present in adult mammals treated in accordance with methods of the invention will become more responsive to cues, e.g., activity cues, which promote the formation and maintenance of synapses.

Performance of methods of the invention, e.g., as described above, may manifest as improvements in observed synaptic plasticity, both in vitro and in vivo as an induction of long-term potentiation. For example, the induction of LTP in neural circuits may be observed in awake individuals, e.g., by performing non-invasive stimulation techniques on awake individuals to induce LTP-like long-lasting changes in localized neural activity (Cooke S F, Bliss T V (2006) Plasticity in the human central nervous system. Brain. 129(Pt 7):1659-73); mapping plasticity and increased neural circuit activity in individuals, e.g., by using positron emission tomography, functional magnetic resonance imaging, and/or transcranial magnetic stimulation (Cramer and Bastings, “Mapping clinically relevant plasticity after stroke,” Neuropharmacology (2000)39:842-51); and by detecting neural plasticity following learning, i.e., improvements in memory, e.g., by assaying retrieval-related brain activity (Buchmann et al., “Prion protein M129V polymorphism affects retrieval-related brain activity,” Neuropsychologia. (2008) 46:2389-402) or, e.g., by imaging brain tissue by functional magnetic resonance imaging (fMRI) following repetition priming with familiar and unfamiliar objects (Soldan et al., “Global familiarity of visual stimuli affects repetition-related neural plasticity but not repetition priming,” Neuroimage. (2008) 39:515-26; Soldan et al., “Aging does not affect brain patterns of repetition effects associated with perceptual priming of novel objects,” J. Cogn. Neurosci. (2008) 20:1762-76). In some embodiments, the method includes the step of measuring synaptic plasticity, and detecting a decreased rate of loss of synaptic plasticity, a stabilization of synaptic plasticity, and/or an increase in synaptic plasticity after administration of the blood product as compared to the synaptic plasticity of the individual before the blood product was administered. Such measurements may be made a week or more after administration of the blood product, e.g., 1 week, 2 weeks, 3 weeks, or more, for instance, 4 weeks, 6 weeks, or 8 weeks or more, e.g., 3 months, 4 months, 5 months, or 6 months or more.

In some instances, the methods result in a change in expression levels of one or more genes in one or more tissues of the host, e.g., as compared to a suitable control (such as described in the Experimental section, below). The change in expression level of a given gene may be 0.5-fold or greater, such as 1.0-fold or greater, including 1.5-fold or greater. The tissue may vary, and in some instances is nervous system tissue, e.g., central nervous system tissue, including brain tissue, e.g., hippocampal tissue. In some instances, the modulation of hippocampal gene expression is manifested as enhanced hippocampal plasticity, e.g., as compared to a suitable control.

In some instances, treatment results in an enhancement in the levels of one or more proteins in one or more tissues of the host, e.g., as compared to a suitable control (such as described in the Experimental section, below). The change in protein level of a given protein may be 0.5 fold or greater, such as 1.0 fold or greater, including 1.5 fold or greater, where in some instances the level may approach that of a healthy wild-type level, e.g., within 50% or less, such as 25% or less, including 10% or less, e.g., 5% or less of the healthy wild-type level. The tissue may vary, and in some instances is nervous system tissue, e.g., central nervous system tissue, including brain tissue, e.g., hippocampal tissue.

In some instances, the methods result in one or more structural changes in one or more tissues. The tissue may vary, and in some instances is nervous system tissue, e.g., central nervous system tissue, including brain tissue, e.g., hippocampal tissue. Structure changes of interest include an increase in dendritic spine density of mature neurons in the dentate gyrus (DG) of the hippocampus, e.g., as compared to a suitable control. In some instances, the modulation of hippocampal structure is manifested as enhanced synapse formation, e.g., as compared to a suitable control. In some instances, the methods may result in an enhancement of long-term potentiation, e.g., as compared to a suitable control.

In some instances, practice of the methods, e.g., as described above, results in an increase in neurogenesis in the adult mammal. The increase may be identified in a number of different ways, e.g., as described below in the Experimental section. In some instances, the increase in neurogenesis manifests as an increase the amount of Dcx-positive immature neurons, e.g., where the increase may be 2-fold or greater. In some instances, the increase in neurogenesis manifests as an increase in the number of BrdU/NeuN positive cells, where the increase may be 2-fold or greater.

In some instances, the methods result in enhancement in learning and memory, e.g., as compared to a suitable control. Enhancement in learning and memory may be evaluated in a number of different ways, e.g., the contextual fear conditioning and/or radial arm water maze (RAWM) paradigms described in the experimental section, below. When measured by contextual fear conditioning, treatment results in some instances in increased freezing in contextual, but not cued, memory testing. When measured by RAWM, treatment results in some instances in enhanced learning and memory for platform location during the testing phase of the task. In some instances, treatment is manifested as enhanced cognitive improvement in hippocampal-dependent learning and memory, e.g., as compared to a suitable control.

In some embodiments, TFF2 level reduction, e.g., as described above, may be performed in conjunction with an active agent having activity suitable to treat aging associated cognitive impairment. For example, a number of active agents have been shown to have some efficacy in treating the cognitive symptoms of Alzheimer's disease (e.g., memory loss, confusion, and problems with thinking and reasoning), e.g., cholinesterase inhibitors (e.g., Donepezil, Rivastigmine, Galantamine, Tacrine), Memantine, and Vitamin E. As another example, a number of agents have been shown to have some efficacy in treating behavioral or psychiatric symptoms of Alzheimer's Disease, e.g., citalopram (Celexa), fluoxetine (Prozac), paroxeine (Paxil), sertraline (Zoloft), trazodone (Desyrel), lorazepam (Ativan), oxazepam (Serax), aripiprazole (Abilify), clozapine (Clozaril), haloperidol (Haldol), olanzapine (Zyprexa), quetiapine (Seroquel), risperidone (Risperdal), and ziprasidone (Geodon).

In some aspects of the subject methods, the method further comprises the step of measuring cognition and/or synaptic plasticity after treatment, e.g., using the methods described herein or known in the art, and determining that the rate of cognitive decline or loss of synaptic plasticity have been reduced and/or that cognitive ability or synaptic plasticity have improved in the individual. In some such instances, the determination is made by comparing the results of the cognition or synaptic plasticity test to the results of the test performed on the same individual at an earlier time, e.g., 2 weeks earlier, 1 month earlier, 2 months earlier, 3 months earlier, 6 months earlier, 1 year earlier, 2 years earlier, 5 years earlier, or 10 years earlier, or more.

In some embodiments, the subject methods further include diagnosing an individual as having a cognitive impairment, e.g., using the methods described herein or known in the art for measuring cognition and synaptic plasticity, prior to administering the subject plasma comprising blood product. In some instances, the diagnosing will comprise measuring cognition and/or synaptic plasticity and comparing the results of the cognition or synaptic plasticity test to one or more references, e.g., a positive control and/or a negative control. For example, the reference may be the results of the test performed by one or more age matched individuals that experience aging-associated cognitive impairments (i.e., positive controls) or that do not experience aging-associated cognitive impairments (i.e., negative controls). As another example, the reference may be the results of the test performed by the same individual at an earlier time, e.g., 2 weeks earlier, 1 month earlier, 2 months earlier, 3 months earlier, 6 months earlier, 1 year earlier, 2 years earlier, 5 years earlier, or 10 years earlier, or more.

In some embodiments, the subject methods further comprise diagnosing an individual as having an aging-associated disorder, e.g., Alzheimer's disease, Parkinson's disease, frontotemporal dementia, progressive supranuclear palsy, Huntington's disease, amyotrophic lateral sclerosis, spinal muscular atrophy, multiple sclerosis, multi-system atrophy, glaucoma, ataxias, myotonic dystrophy, dementia, and the like. Methods for diagnosing such aging-associated disorders are well-known in the art, any of which may be used by the ordinarily skilled artisan in diagnosing the individual. In some embodiments, the subject methods further comprise both diagnosing an individual as having an aging associated disorder and as having a cognitive impairment.

### 9. UTILITY

The subject methods find use in treating, including preventing, aging-associated impairments and conditions associated therewith, such as impairments in the cognitive ability of individuals. Individuals suffering from or at risk of developing an aging-associated cognitive impairments include individuals that are about 50 years old or older, e.g., 60 years old or older, 70 years old or older, 80 years old or older, 90 years old or older, and usually no older than 100 years old, i.e., between the ages of about 50 and 100, e.g., 50, 55, 60, 65, 70, 75, 80, 85, 90, 95 or about 100 years old, and are suffering from cognitive impairment associated with natural aging process, e.g., mild cognitive impairment (M.C.I.); and individuals that are about 50 years old or older, e.g., 60 years old or older, 70 years old or older, 80 years old or older, 90 years old or older, and usually no older than 100 years old, i.e., between the ages of about 50 and 90, e.g., 50, 55, 60, 65, 70, 75, 80, 85, 90, 95 or about 100 years old, that have not yet begun to show symptoms of cognitive impairment. Examples of cognitive impairments that are due to natural aging include the following:

Mild cognitive impairment (M.C.I.) is a modest disruption of cognition that manifests as problems with memory or other mental functions such as planning, following instructions, or making decisions that have worsened over time while overall mental function and daily activities are not impaired. Thus, although significant neuronal death does not typically occur, neurons in the aging brain are vulnerable to sub-lethal age-related alterations in structure, synaptic integrity, and molecular processing at the synapse, all of which impair cognitive function.

Individuals suffering from or at risk of developing an aging-associated cognitive impairment that will benefit from treatment with the subject plasma-comprising blood product, e.g., by the methods disclosed herein, also include individuals of any age that are suffering from a cognitive impairment due to an aging-associated disorder; and individuals of any age that have been diagnosed with an aging-associated disorder that is typically accompanied by cognitive impairment, where the individual has not yet begun to present with symptoms of cognitive impairment. Examples of such aging-associated disorders include the following:

Alzheimer's disease (AD). Alzheimer's disease is a progressive, inexorable loss of cognitive function associated with an excessive number of senile plaques in the cerebral cortex and subcortical gray matter, which also contains b-amyloid and neurofibrillary tangles consisting of tau protein. The common form affects persons >60 yr. old, and its incidence increases as age advances. It accounts for more than 65% of the dementias in the elderly.

The cause of Alzheimer's disease is not known. The disease runs in families in about 15 to 20% of cases. The remaining, so-called sporadic cases have some genetic determinants. The disease has an autosomal dominant genetic pattern in most early-onset and some late-onset cases but a variable late-life penetrance. Environmental factors are the focus of active investigation.

In the course of the disease, synapses, and ultimately neurons are lost within the cerebral cortex, hippocampus, and subcortical structures (including selective cell loss in the nucleus basalis of Meynert), locus caeruleus, and nucleus raphae dorsalis. Cerebral glucose use and perfusion is reduced in some areas of the brain (parietal lobe and temporal cortices in early-stage disease, prefrontal cortex in late-stage disease). Neuritic or senile plaques (composed of neurites, astrocytes, and glial cells around an amyloid core) and neurofibrillary tangles (composed of paired helical filaments) play a role in the pathogenesis of Alzheimer's disease. Senile plaques and neurofibrillary tangles occur with normal aging, but they are much more prevalent in persons with Alzheimer's disease.

Parkinson's Disease. Parkin son's Disease (PD) is an idiopathic, slowly progressive, degenerative CNS disorder characterized by slow and decreased movement, muscular rigidity, resting tremor, and postural instability. Originally considered primarily a motor disorder, PD is now recognized to also affect cognition, behavior, sleep, autonomic function, and sensory function. The most common cognitive impairments include an impairment in attention and concentration, working memory, executive function, producing language, and visuospatial function.

In primary Parkinson's disease, the pigmented neurons of the substantia nigra, locus caeruleus, and other brain stem dopaminergic cell groups are lost. The cause is not known. The loss of substantia nigra neurons, which project to the caudate nucleus and putamen, results in depletion of the neurotransmitter dopamine in these areas. Onset is generally after age 40, with increasing incidence in older age groups.

Secondary parkinsonism results from loss of or interference with the action of dopamine in the basal ganglia due to other idiopathic degenerative diseases, drugs, or exogenous toxins. The most common cause of secondary parkinsonism is ingestion of antipsychotic drugs or reserpine, which produce parkinsonism by blocking dopamine receptors. Less common causes include carbon monoxide or manganese poisoning, hydrocephalus, structural lesions (tumors, infarcts affecting the midbrain or basal ganglia), subdural hematoma, and degenerative disorders, including striatonigral degeneration.

Frontotemporal dementia. Frontotemporal dementia (FTD) is a condition resulting from the progressive deterioration of the frontal lobe of the brain. Over time, the degeneration may advance to the temporal lobe. Second only to Alzheimer's disease (AD) in prevalence, FTD accounts for 20% of pre-senile dementia cases. Symptoms are classified into three groups based on the functions of the frontal and temporal lobes affected: Behavioral variant FTD (bvFTD), with symptoms include lethargy and aspontaneity on the one hand, and disinhibition on the other; progressive nonfluent aphasia (PNFA), in which a breakdown in speech fluency due to articulation difficulty, phonological and/or syntactic errors is observed but word comprehension is preserved; and semantic dementia (SD), in which patients remain fluent with normal phonology and syntax but have increasing difficulty with naming and word comprehension. Other cognitive symptoms common to all FTD patients include an impairment in executive function and ability to focus. Other cognitive abilities, including perception, spatial skills, memory and praxis typically remain intact. FTD can be diagnosed by observation of reveal frontal lobe and/or anterior temporal lobe atrophy in structural MRI scans.

A number of forms of FTD exist, any of which may be treated or prevented using the subject methods and compositions. For example, one form of frontotemporal dementia is Semantic Dementia (SD). SD is characterized by a loss of semantic memory in both the verbal and non-verbal domains. SD patients often present with the complaint of word-finding difficulties. Clinical signs include fluent aphasia, anomia, impaired comprehension of word meaning, and associative visual agnosia (the inability to match semantically related pictures or objects). As the disease progresses, behavioral and personality changes are often seen similar to those seen in frontotemporal dementia although cases have been described of ‘pure’ semantic dementia with few late behavioral symptoms. Structural MRI imaging shows a characteristic pattern of atrophy in the temporal lobes (predominantly on the left), with inferior greater than superior involvement and anterior temporal lobe atrophy greater than posterior.

As another example, another form of frontotemporal dementia is Pick's disease (PiD, also PcD). A defining characteristic of the disease is build-up of tau proteins in neurons, accumulating into silver-staining, spherical aggregations known as “Pick bodies”. Symptoms include loss of speech (aphasia) and dementia. Patients with orbitofrontal dysfunction can become aggressive and socially inappropriate. They may steal or demonstrate obsessive or repetitive stereotyped behaviors. Patients with dorsomedial or dorsolateral frontal dysfunction may demonstrate a lack of concern, apathy, or decreased spontaneity. Patients can demonstrate an absence of self-monitoring, abnormal self-awareness, and an inability to appreciate meaning. Patients with gray matter loss in the bilateral posterolateral orbitofrontal cortex and right anterior insula may demonstrate changes in eating behaviors, such as a pathologic sweet tooth. Patients with more focal gray matter loss in the anterolateral orbitofrontal cortex may develop hyperphagia. While some of the symptoms can initially be alleviated, the disease progresses, and patients often die within two to ten years.

Huntington's disease. Huntington's disease (HD) is a hereditary progressive neurodegenerative disorder characterized by the development of emotional, behavioral, and psychiatric abnormalities; loss of intellectual or cognitive functioning; and movement abnormalities (motor disturbances). The classic signs of HD include the development of chorea—involuntary, rapid, irregular, jerky movements that may affect the face, arms, legs, or trunk—as well as cognitive decline including the gradual loss of thought processing and acquired intellectual abilities. There may be impairment of memory, abstract thinking, and judgment; improper perceptions of time, place, or identity (disorientation); increased agitation; and personality changes (personality disintegration). Although symptoms typically become evident during the fourth or fifth decades of life, the age at onset is variable and ranges from early childhood to late adulthood (e.g., 70s or 80s).

HD is transmitted within families as an autosomal dominant trait. The disorder occurs as the result of abnormally long sequences or “repeats” of coded instructions within a gene on chromosome 4 (4p16.3). The progressive loss of nervous system function associated with HD results from loss of neurons in certain areas of the brain, including the basal ganglia and cerebral cortex.

Amyotrophic lateral sclerosis. Amyotrophic lateral sclerosis (ALS) is a rapidly progressive, invariably fatal neurological disease that attacks motor neurons. Muscular weakness and atrophy and signs of anterior horn cell dysfunction are initially noted most often in the hands and less often in the feet. The site of onset is random, and progression is asymmetric. Cramps are common and may precede weakness. Rarely, a patient survives 30 years; 50% die within 3 years of onset, 20% live 5 years, and 10% live 10 years. Diagnostic features include onset during middle or late adult life and progressive, generalized motor involvement without sensory abnormalities. Nerve conduction velocities are normal until late in the disease. Recent studies have documented the presentation of cognitive impairments as well, particularly a reduction in immediate verbal memory, visual memory, language, and executive function.

A decrease in cell body area, number of synapses and total synaptic length has been reported in even normal-appearing neurons of the ALS patients. It has been suggested that when the plasticity of the active zone reaches its limit, a continuing loss of synapses can lead to functional impairment. Promoting the formation or new synapses or preventing synapse loss may maintain neuron function in these patients.

Multiple Sclerosis. Multiple Sclerosis (MS) is characterized by various symptoms and signs of CNS dysfunction, with remissions and recurring exacerbations. The most common presenting symptoms are paresthesias in one or more extremities, in the trunk, or on one side of the face; weakness or clumsiness of a leg or hand; or visual disturbances, e.g., partial blindness and pain in one eye (retrobulbar optic neuritis), dimness of vision, or scotomas. Common cognitive impairments include impairments in memory (acquiring, retaining, and retrieving new information), attention and concentration (particularly divided attention), information processing, executive functions, visuospatial functions, and verbal fluency. Common early symptoms are ocular palsy resulting in double vision (diplopia), transient weakness of one or more extremities, slight stiffness or unusual fatigability of a limb, minor gait disturbances, difficulty with bladder control, vertigo, and mild emotional disturbances; all indicate scattered CNS involvement and often occur months or years before the disease is recognized. Excess heat may accentuate symptoms and signs.

The course is highly varied, unpredictable, and, in most patients, remittent. At first, months or years of remission may separate episodes, especially when the disease begins with retrobulbar optic neuritis. However, some patients have frequent attacks and are rapidly incapacitated; for a few the course can be rapidly progressive.

Glaucoma. Glaucoma is a common neurodegenerative disease that affects retinal ganglion cells (RGCs). Evidence supports the existence of compartmentalized degeneration programs in synapses and dendrites, including in RGCs. Recent evidence also indicates a correlation between cognitive impairment in older adults and glaucoma (Yochim B P, et al. Prevalence of cognitive impairment, depression, and anxiety symptoms among older adults with glaucoma. J Glaucoma. 2012; 21(4):250-254).

Myotonic dystrophy. Myotonic dystrophy (DM) is an autosomal dominant multisystem disorder characterized by dystrophic muscle weakness and myotonia. The molecular defect is an expanded trinucleotide (CTG) repeat in the 3′ untranslated region of the myotonin-protein kinase gene on chromosome 19q. Symptoms can occur at any age, and the range of clinical severity is broad. Myotonia is prominent in the hand muscles, and ptosis is common even in mild cases. In severe cases, marked peripheral muscular weakness occurs, often with cataracts, premature balding, hatchet facies, cardiac arrhythmias, testicular atrophy, and endocrine abnormalities (e.g., diabetes mellitus). Mental retardation is common in severe congenital forms, while an aging-related decline of frontal and temporal cognitive functions, particularly language and executive functions, is observed in milder adult forms of the disorder. Severely affected persons die by their early 50s.

Dementia. Dementia describes class of disorders having symptoms affecting thinking and social abilities severely enough to interfere with daily functioning. Other instances of dementia in addition to the dementia observed in later stages of the aging associated disorders discussed above include vascular dementia, and dementia with Lewy bodies, described below.

In vascular dementia, or “multi-infarct dementia”, cognitive impairment is caused by problems in supply of blood to the brain, typically by a series of minor strokes, or sometimes, one large stroke preceded or followed by other smaller strokes. Vascular lesions can be the result of diffuse cerebrovascular disease, such as small vessel disease, or focal lesions, or both. Patients suffering from vascular dementia present with cognitive impairment, acutely or subacutely, after an acute cerebrovascular event, after which progressive cognitive decline is observed. Cognitive impairments are similar to those observed in Alzheimer's disease, including impairments in language, memory, complex visual processing, or executive function, although the related changes in the brain are not due to AD pathology but to chronic reduced blood flow in the brain, eventually resulting in dementia. Single photon emission computed tomography (SPECT) and positron emission tomography (PET) neuroimaging may be used to confirm a diagnosis of multi-infarct dementia in conjunction with evaluations involving mental status examination.

Dementia with Lewy bodies (DLB, also known under a variety of other names including Lewy body dementia, diffuse Lewy body disease, cortical Lewy body disease, and senile dementia of Lewy type) is a type of dementia characterized anatomically by the presence of Lewy bodies (clumps of alpha-synuclein and ubiquitin protein) in neurons, detectable in post mortem brain histology. Its primary feature is cognitive decline, particularly of executive functioning. Alertness and short-term memory will rise and fall. Persistent or recurring visual hallucinations with vivid and detailed pictures are often an early diagnostic symptom. DLB it is often confused in its early stages with Alzheimer's disease and/or vascular dementia, although, where Alzheimer's disease usually begins quite gradually, DLB often has a rapid or acute onset. DLB symptoms also include motor symptoms similar to those of Parkinson's. DLB is distinguished from the dementia that sometimes occurs in Parkinson's disease by the time frame in which dementia symptoms appear relative to Parkinson symptoms. Parkinson's disease with dementia (PDD) would be the diagnosis when dementia onset is more than a year after the onset of Parkinson's. DLB is diagnosed when cognitive symptoms begin at the same time or within a year of Parkinson symptoms.

Progressive supranuclear palsy. Progressive supranuclear palsy (PSP) is a brain disorder that causes serious and progressive problems with control of gait and balance, along with complex eye movement and thinking problems. One of the classic signs of the disease is an inability to aim the eyes properly, which occurs because of lesions in the area of the brain that coordinates eye movements. Some individuals describe this effect as a blurring. Affected individuals often show alterations of mood and behavior, including depression and apathy as well as progressive mild dementia. The disorder's long name indicates that the disease begins slowly and continues to get worse (progressive), and causes weakness (palsy) by damaging certain parts of the brain above pea-sized structures called nuclei that control eye movements (supranuclear). PSP was first described as a distinct disorder in 1964, when three scientists published a paper that distinguished the condition from Parkinson's disease. It is sometimes referred to as Steele-Richardson-Olszewski syndrome, reflecting the combined names of the scientists who defined the disorder. Although PSP gets progressively worse, no one dies from PSP itself.

Ataxia. People with ataxia have problems with coordination because parts of the nervous system that control movement and balance are affected. Ataxia may affect the fingers, hands, arms, legs, body, speech, and eye movements. The word ataxia is often used to describe a symptom of incoordination which can be associated with infections, injuries, other diseases, or degenerative changes in the central nervous system. Ataxia is also used to denote a group of specific degenerative diseases of the nervous system called the hereditary and sporadic ataxias which are the National Ataxia Foundation's primary emphases.

Multiple-system atrophy. Multiple-system atrophy (MSA) is a degenerative neurological disorder. MSA is associated with the degeneration of nerve cells in specific areas of the brain. This cell degeneration causes problems with movement, balance, and other autonomic functions of the body such as bladder control or blood-pressure regulation. The cause of MSA is unknown and no specific risk factors have been identified. Around 55% of cases occur in men, with typical age of onset in the late 50s to early 60s. MSA often presents with some of the same symptoms as Parkinson's disease. However, MSA patients generally show minimal if any response to the dopamine medications used for Parkinson's.

Frailty. Frailty Syndrome (“Frailty”) is a geriatric syndrome characterized by functional and physical decline including decreased mobility, muscle weakness, physical slowness, poor endurance, low physical activity, malnourishment, and involuntary weight loss. Such decline is often accompanied and a consequence of diseases such as cognitive dysfunction and cancer. However, Frailty can occur even without disease. Individuals suffering from Frailty have an increased risk of negative prognosis from fractures, accidental falls, disability, comorbidity, and premature mortality. (C. Buigues, et al. Effect of a Prebiotic Formulation on Frailty Syndrome: A Randomized, Double-Blind Clinical Trial, Int. J. Mol. Sci. 2016, 17, 932). Additionally, individuals suffering from Frailty have an increased incidence of higher health care expenditure. (Id.)

Common symptoms of Frailty can be determined by certain types of tests. For example, unintentional weight loss involves a loss of at least 10 lbs. or greater than 5% of body weight in the preceding year; muscle weakness can be determined by reduced grip strength in the lowest 20% at baseline (adjusted for gender and BMI); physical slowness can be based on the time needed to walk a distance of 15 feet; poor endurance can be determined by the individual's self-reporting of exhaustion; and low physical activity can be measured using a standardized questionnaire. (Z. Palace et al., The Frailty Syndrome, Today's Geriatric Medicine 7(1), at 18 (2014)).

In some embodiments, the subject methods and compositions find use in slowing the progression of aging-associated cognitive impairment. In other words, cognitive abilities in the individual will decline more slowly following treatment by the disclosed methods than prior to or in the absence of treatment by the disclosed methods. In some such instances, the subject methods of treatment include measuring the progression of cognitive decline after treatment and determining that the progression of cognitive decline is reduced. In some such instances, the determination is made by comparing to a reference, e.g., the rate of cognitive decline in the individual prior to treatment, e.g., as determined by measuring cognition prior at two or more time points prior to administration of the subject blood product.

The subject methods and compositions also find use in stabilizing the cognitive abilities of an individual, e.g., an individual suffering from aging-associated cognitive decline or an individual at risk of suffering from aging-associated cognitive decline. For example, the individual may demonstrate some aging-associated cognitive impairment, and progression of cognitive impairment observed prior to treatment with the disclosed methods will be halted following treatment by the disclosed methods. As another example, the individual may be at risk for developing an aging-associated cognitive decline (e.g., the individual may be aged 50 years old or older, or may have been diagnosed with an aging-associated disorder), and the cognitive abilities of the individual are substantially unchanged, i.e., no cognitive decline can be detected, following treatment by the disclosed methods as compared to prior to treatment with the disclosed methods.

The subject methods and compositions also find use in reducing cognitive impairment in an individual suffering from an aging-associated cognitive impairment. In other words, cognitive ability is improved in the individual following treatment by the subject methods. For example, the cognitive ability in the individual is increased, e.g., by 2-fold or more, 5-fold or more, 10-fold or more, 15-fold or more, 20-fold or more, 30-fold or more, or 40-fold or more, including 50-fold or more, 60-fold or more, 70-fold or more, 80-fold or more, 90-fold or more, or 100-fold or more, following treatment by the subject methods relative to the cognitive ability that is observed in the individual prior to treatment by the subject methods. In some instances, treatment by the subject methods and compositions restores the cognitive ability in the individual suffering from aging-associated cognitive decline, e.g., to their level when the individual was about 40 years old or less. In other words, cognitive impairment is abrogated.

### 10. REAGENTS, DEVICES AND KITS

Also provided are reagents, devices and kits thereof for practicing one or more of the above-described methods. The subject reagents, devices and kits thereof may vary greatly. Reagents and devices of interest include those mentioned above with respect to the methods of reducing TFF2 levels in an adult mammal and the methods of attenuating the levels or activity of TFF2 in the subject diagnosed with a age-related disorder, or cognitive impairment.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, portable flash drive, etc., on which the information has been recorded. Yet another means that may be present is a website address which may be used via the internet to access the information at a removed site. Any convenient means may be present in the kits.

### 11. EXAMPLES

The following examples are provided by way of illustration and not by way of limitation.

### A. Experimental Examples

i. TFF2 Levels Increase with Age

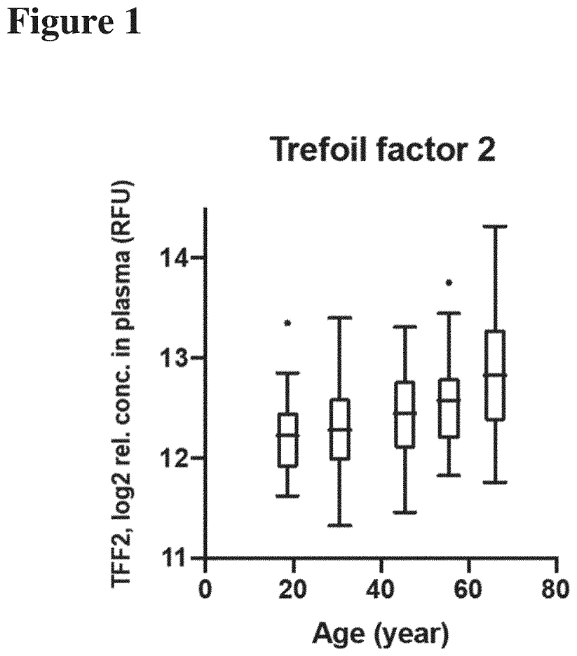

FIG. 1 shows a “box and whiskers” depiction of the log 2 relative concentrations of TFF2 in plasma from donors of five different age groups. Plasma from males (50 individuals in each age group) aged 18, 30, 45, 55, and 66-years-old were measured using the SomaScan aptamer-based proteomics assay (SomaLogic, Boulder, Colo.). Healthy plasma levels show a highly significant monotonous increase over this age range (p=1.6e-9, Jonckheere-Terpstra trend test). The line within each box indicates the median value.

ii. Effect of Human Recombinant TFF2 Protein in Young C57BL/6 Mice

Three-month-old C57BL6 mice were treated with recombinant human TFF2 (“hTFF2,” 1.25 μg/mouse, IP) or vehicle (PBS) every other day for 4 weeks (n=14-15 per group). Mice were tested in a set of behavior assays, and brains subsequently analyzed.

FIG. 1 [[differential relative quantification in old and young plasma]]

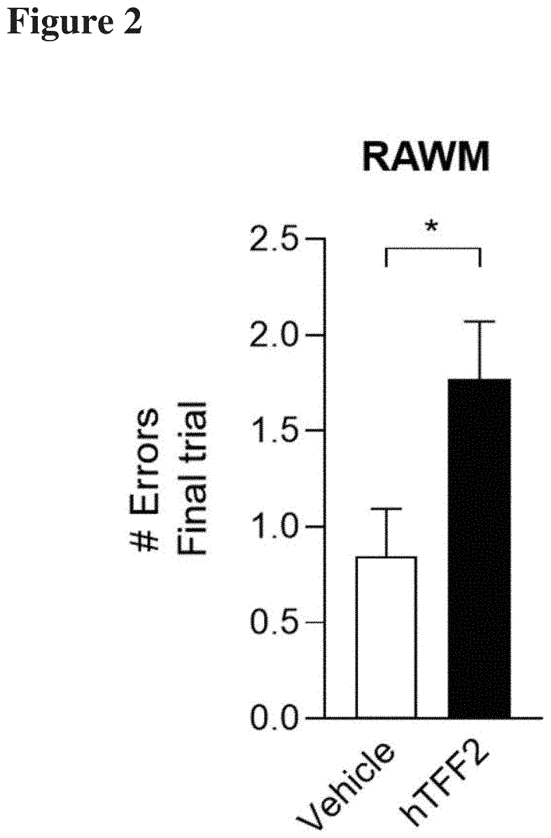

FIG. 2 shows the results of a radial arm water maze (RAWM) assay which tests reference and working memory performance by requiring the mice to utilize cues to locate escape platforms. (See, e.g., Penley S C, et al., J Vis Exp., (82):50940 (2013)). Young mice treated with hTFF2 made more errors when navigating the maze compared to vehicle-treated mice.

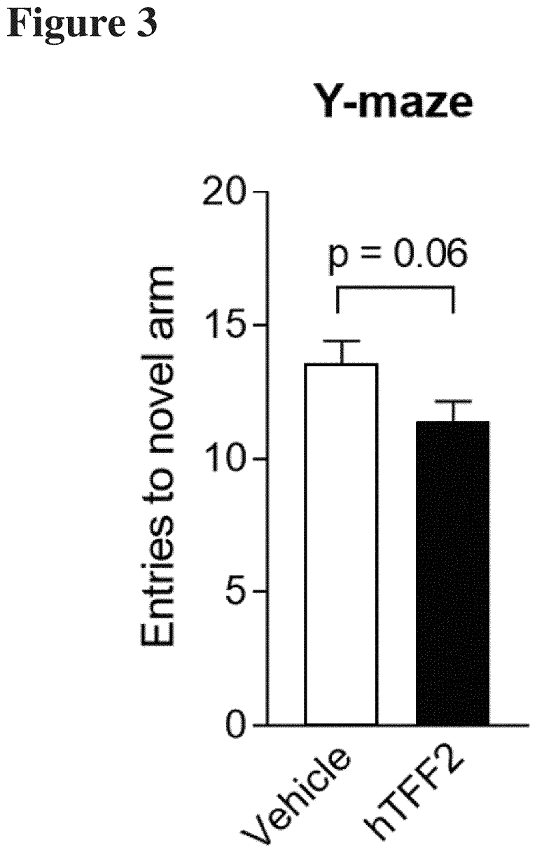

FIG. 3 depicts the results from a Y-maze behavior test. The Y-maze test determines hippocampal-dependent cognition as measured by preference to enter the novel arm (as opposed to the familiar arm) in a cued Y-maze. The percent entries were calculated by normalizing the number of entries in the novel or familiar arm (the two arms of the “Y” maze) to the total entries in the novel and familiar arms. The Wilcoxon matched pairs signed rank test was used to assess statistical significance between novel and familiar arms in percent of entries. The results of FIG. 3 demonstrate that administration of human TFF2 (hTFF2) to young mice leads to a trend of fewer entries into the novel arm of the Y-maze, indicating a decline in cognitive performance.

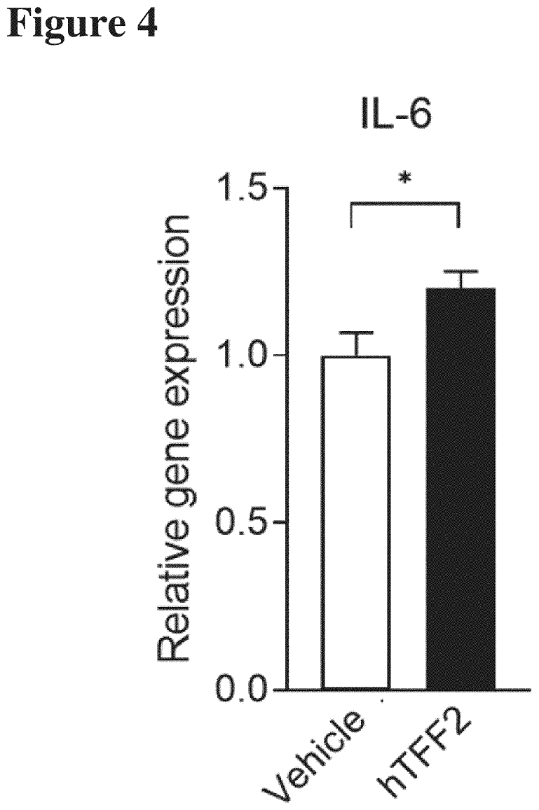

FIG. 4 shows quantitative PCR (qPCR) of hippocampal mRNA from hTFF2-treated and vehicle-treated mice. The figure shows that there is an increase in expression of an inflammatory marker, IL-6, as compared to vehicle treated mice. (* P<0.05, Mann-Whitney U test).

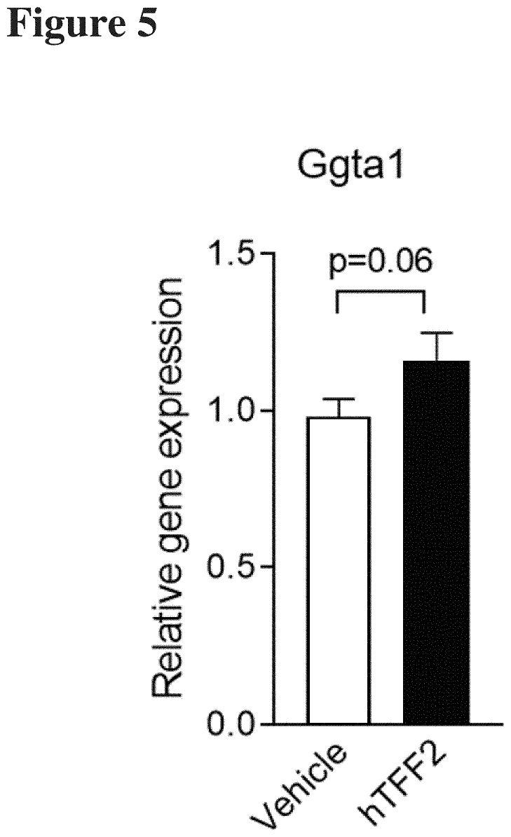

FIG. 5 shows RT-qPCR of hippocampal cDNA from hTFF2- and vehicle-treated mice. The figure shows that there is a trend in increased expression of a marker for reactive astrocytes, Ggta1, as compared to vehicle-treated mice. Reactive astrocytes are strongly induced by the central nervous system during injury and disease. (Liddelow S A, et al., Nature, 541(7638):481-87 (2017).

This data shows that the cognitive performance of young mice can be compromised by the presence of hTFF2, making TFF2 a target for inhibition in cognitive disease or other disorders.

iii. TFF2 Inhibition in 21-Month-Old Mice

Twenty-one-month-old C57BL6 mice were treated with the TFF2 inhibitor, L-pyroglutamic acid (30 mg/kg, daily PO) or vehicle (4% DMSO in sterile Kolliphor/EtOH) for 4 weeks (n=15 per group) and subjected to behavioral testing. Behavioral testing was initiated after 3 weeks of treatment. Mice were sacrificed one day following the conclusion of the last behavior test.

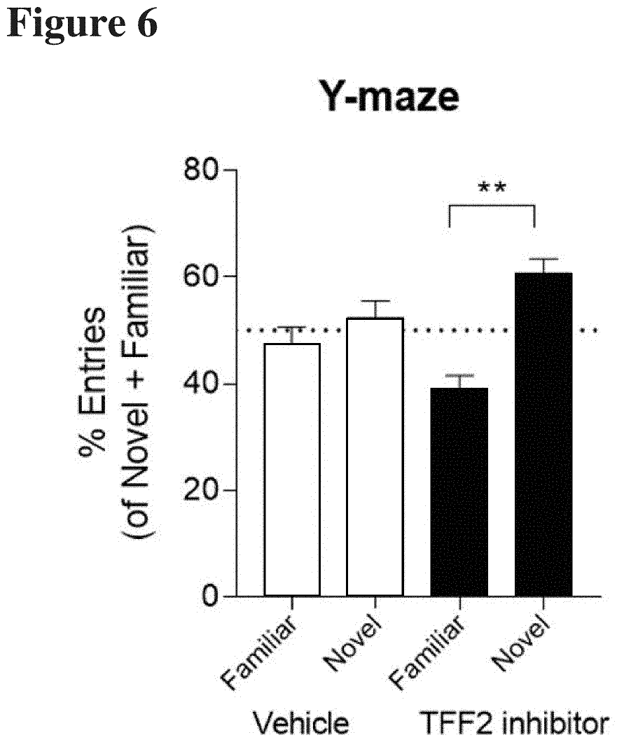

FIG. 6 demonstrates that TFF2 inhibition with L-pyroglutamic acid improved cognitive performance in a Y-maze test as aged mice treated with the inhibitor entered the novel arm significantly more than the familiar arm (p<0.002) and the difference between novel and familiar arm entries was greater than that observed with vehicle. Data is shown as mean±SEM.

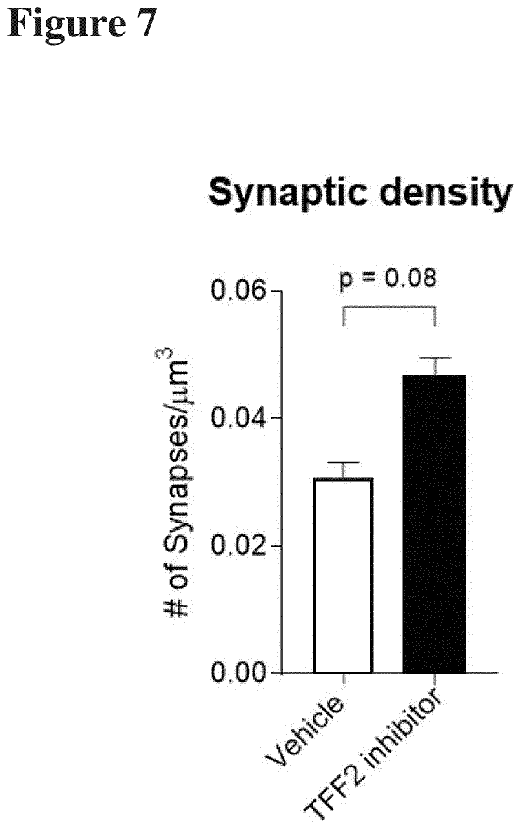

FIG. 7 shows results from quantitative analysis of immunostaining in hippocampi of aged mice treated with the TFF2 inhibitor compared to vehicle. Synapse density was measured as number of synapses per μm³. There was a strong trend towards higher synapse density in the CA1 region of the hippocampus in mice treated with TFF2 inhibitor. Data is shown as mean±SEM.

iv. Effect of Anti-TFF2 Antibodies on TFF2 Activity

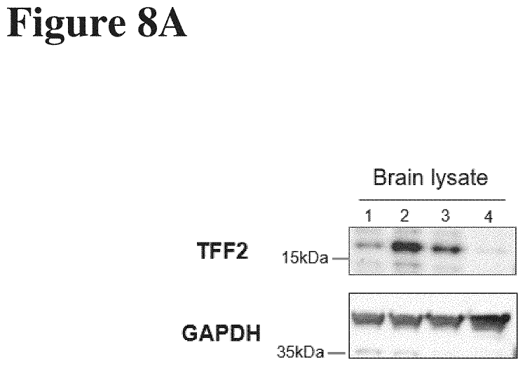

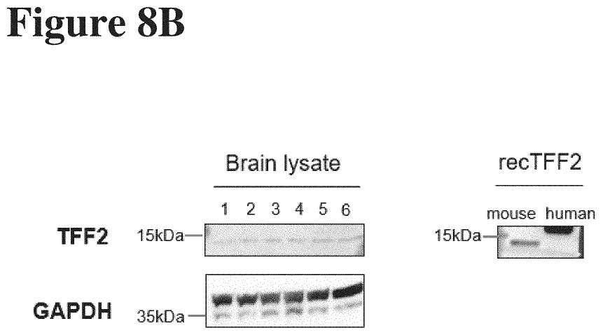

Hemibrains from 22-month-old C57B16 mice were homogenized in PBS with protease inhibitors. Samples from 4-6 mice were probed with a rabbit polyclonal anti-human TFF2 antibody (Life Science Bio, LS-C4895). FIG. 8A is a Western blot demonstrating that TFF2 protein is detected in brain lysate from four 22-month-old mice. FIG. 8B shows that the anti-TFF2 antibody recognizes both mouse and human recombinant TFF2 and that mouse TFF2 (12 kDa) and human TFF2 (14 kDa) can be glycosylated in vivo.

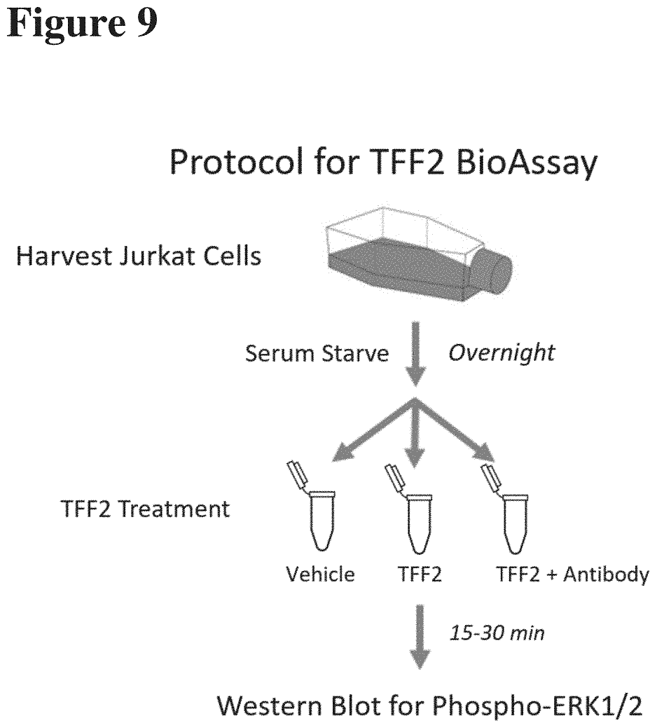

FIG. 9 describes a TFF2 bioassay for ERK1/2 phosphorylation in Jurkat cells (ATCC, TIB-152). Jurkat cells are a human acute T cell leukemia cell line that express CXCR4, a receptor reported to interact with TFF2 and binds to ligand SDF-1. Stimulation of CXCR4 leads to activation of downstream signaling pathways including phosphorylation of ERK1/2. An assay was herein developed to measure TFF2 activation and inhibition in vitro via Western blotting for ERK1/2 phosphorylation. The assay is performed as follows: Jurkat cells are grown in RPMI media with 10% FBS in a T-75 flask to confluency. Cells are counted, and 10⁷ cells are resuspended in in RPMI with no FBS and incubated overnight at 37° C., 5% CO₂. Serum starved cells are counted, and 2×10⁵ cells are added to sample tubes. Cells are treated with vehicle, TFF2, or positive control mouse SDF-1. Anti-TFF2 antibodies to be tested are then added to the cells, and samples are incubated at 37° C., 5% CO₂ for 15-30 min. Cells are lysed in RIPA with protease and phosphatase inhibitors, and lysates are run on a 4-12% Bis-Tris gel in MOPS buffer. After membrane transfer, blots are blocked in 5% BSA and probed with a rabbit anti-phospho ERK1/2 antibody (Cell Signaling Technologies, 4307).

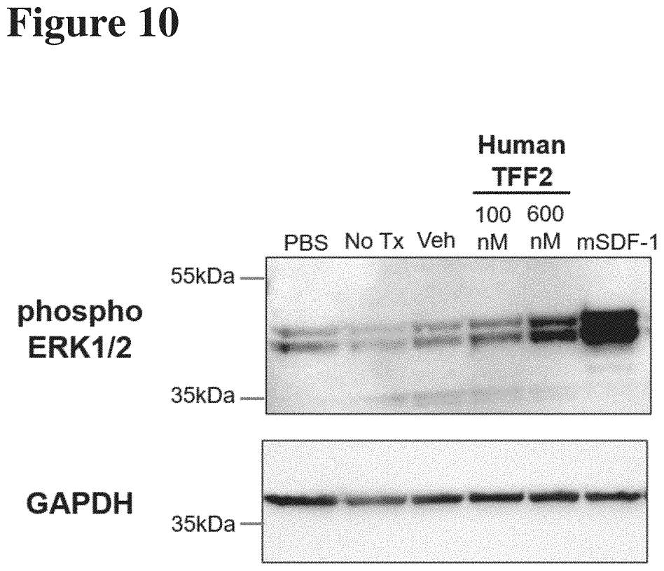

FIG. 10 shows a Western blot demonstrating that treatment of Jurkat cells with human TFF2 leads to increased ERK1/2 phosphorylation. Incubation of Jurkat cells with 100 or 600 nM TFF2 induces ERK1/2 phosphorylation over controls (PBS, no treatment (No Tx), or water (Veh). Positive control mouse SDF-1 (10 g/ml) shows strong ERK1/2 phosphorylation. Housekeeping gene glyceraldehyde 3-phosphate dehydrogenase (GAPDH) was used as a loading control.

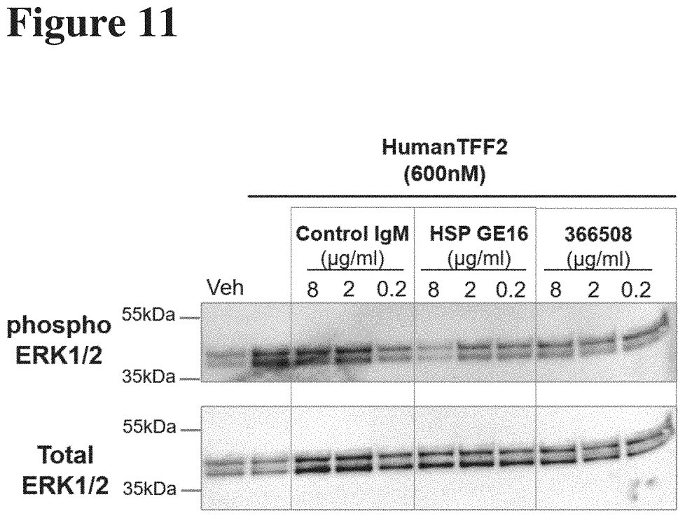

FIG. 11 is a Western blot showing that anti-human TFF2 antibodies have neutralizing activity in Jurkat cells against human TFF2. Two monoclonal anti-human TFF2 antibodies were tested in the TFF2 bioassay for neutralizing activity at different concentrations (8, 2, 0.2 μg/mL). HSPGE16C (R&D Systems) was raised against the last 20 amino acids of TFF2, whereas clone 366508 recognizes a portion of TFF2 (Glu24-Tyr129). An IgM isotype control was used at the same concentrations, but do not inhibit ERK1/2 phosphorylation. HSP GE16 antibody clone shows inhibition at highest concentrations, whereas clone 366508 shows moderate inhibition. Total ERK1/2 was used as a loading control.

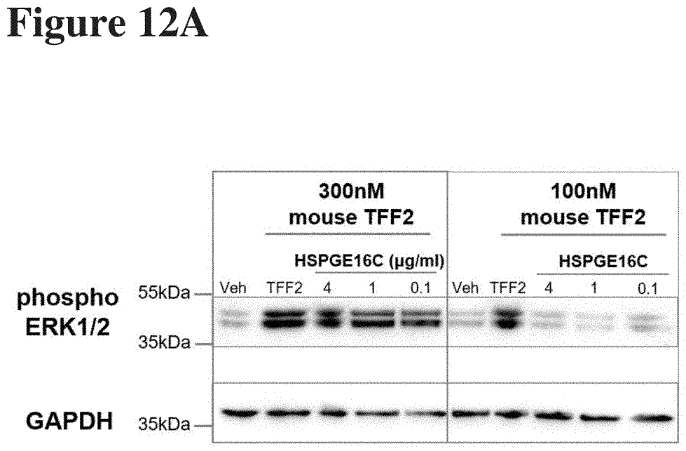

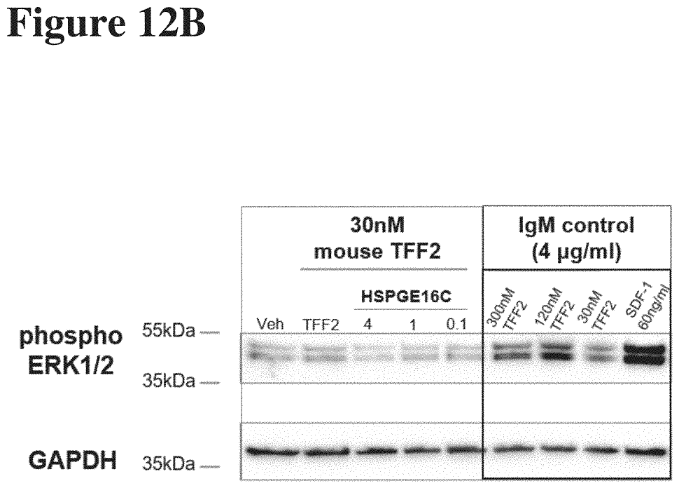

FIGS. 12A and 12B demonstrate that an anti-TFF2 antibody can neutralize mouse TFF2 activity in Jurkat cells. Mouse TFF2 (“TFF2” column) can also induce ERK1/2 phosphorylation in Jurkat cells at higher concentrations (FIG. 12A 300 nM and 100 nM, but not 30 nM TFF2, see FIG. 12B). Anti-human TFF2 antibody clone HSPGE16C can inhibit ERK1/2 phosphorylation with treatment of 100 nM TFF2, but not 300 nM. GAPDH was used as a loading control.

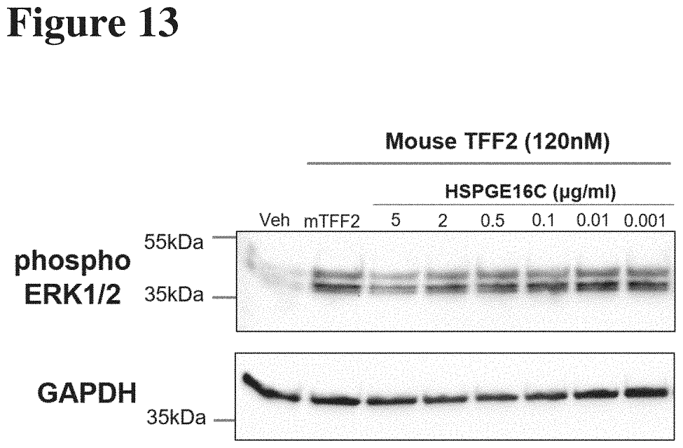

FIG. 13 is a Western blot showing that HSPGE16C anti-hTFF2 antibody can neutralize mouse TFF2 activity in Jurkat cells in a concentration-dependent manner, with a decrease in ERK1/2 phosphorylation at higher concentrations. GAPDH was used as a loading control.

v. TFF2 Antibodies Inhibit TFF2 Activity in Jurkat Cells

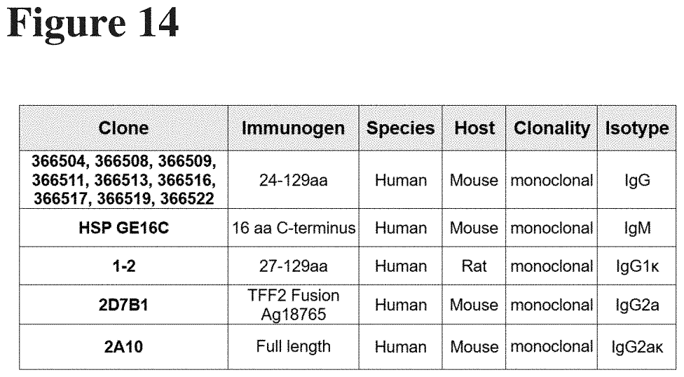

Commercially available anti-TFF2 antibodies were tested for neutralization of TFF2 activity in Jurkat cells. FIG. 14 shows a table of commercially available anti-TFF2 antibodies that were tested for neutralization of TFF2 activity in Jurkat cells, as well as their immunogen information, the species of TFF2 the antibody recognizes, the host species they were produced from, their clonality, and their isotype.

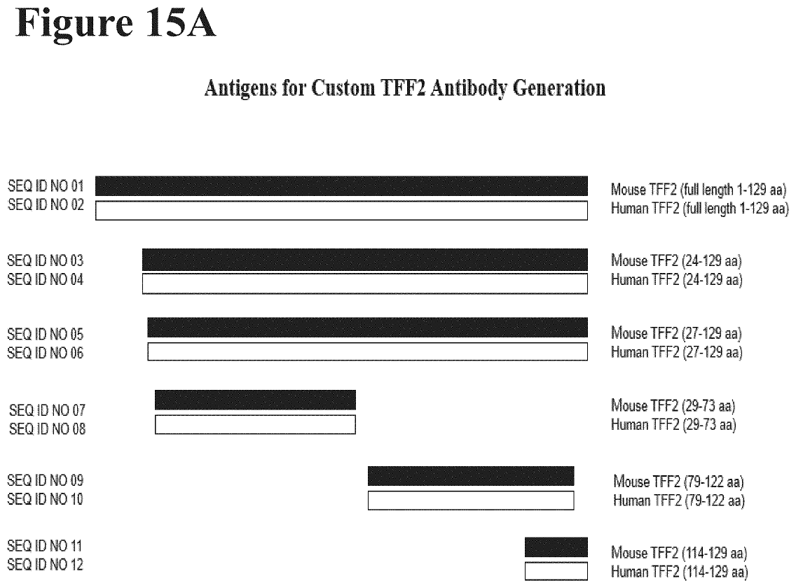

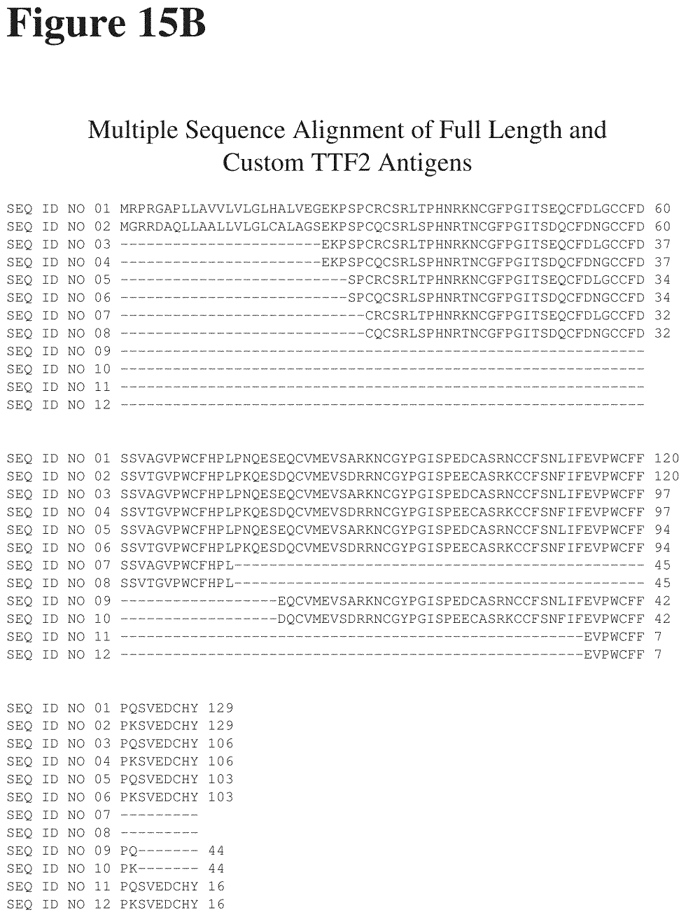

FIG. 15A shows representations of the peptide sequences for full length Mouse TFF2, which is labelled SEQ ID NO: 01, and Human TFF2, which is labelled SEQ ID NO: 02, as well as the TFF2 antigens used to generate antibodies for specific protein domains. Mouse sequences are represented as black rectangles and human sequences as white rectangles with each peptide region aligned with the full length TFF2 proteins. The antigens include amino acids 24-129 of Mouse TFF2 (SEQ ID NO: 03); amino acids 24-129 of Human TFF2 (SEQ ID NO: 04); amino acids 27-129 of Mouse TFF2 (SEQ ID NO: 05); amino acids 27-129 of Human TFF2 (SEQ ID NO: 06); amino acids 29-73 of Mouse TFF2 (SEQ ID NO: 07); amino acids 29-73 of Human TFF2 (SEQ ID NO: 08); amino acids 79-122 of Mouse TFF2 (SEQ ID NO: 09); amino acids 79-122 of Human TFF2 (SEQ ID NO: 10); amino acids 114-129 of Mouse TFF2 (SEQ ID NO: 11); and amino acids 114-129 of Human TFF2 (SEQ ID NO: 12). Different peptide fragments and full-length mouse and human TFF2 are used to generate antibodies that are specific for protein domains. Commercially available antibodies generated from these sequences were screened for specific binding to TFF2 and neutralization in vitro. These antigens can also be used to generate custom TFF2 antibodies and help to identify antigenic regions that result in production of antibodies that are more effective in attenuating TFF2 activity.

FIG. 15B shows a multiple sequence alignment of SEQ ID NOs 1 through 12 described in FIG. 15A. The alignment was performed using CLUSTAL 0 (1.2.4) (available at https://www.uniprot.org/align/).

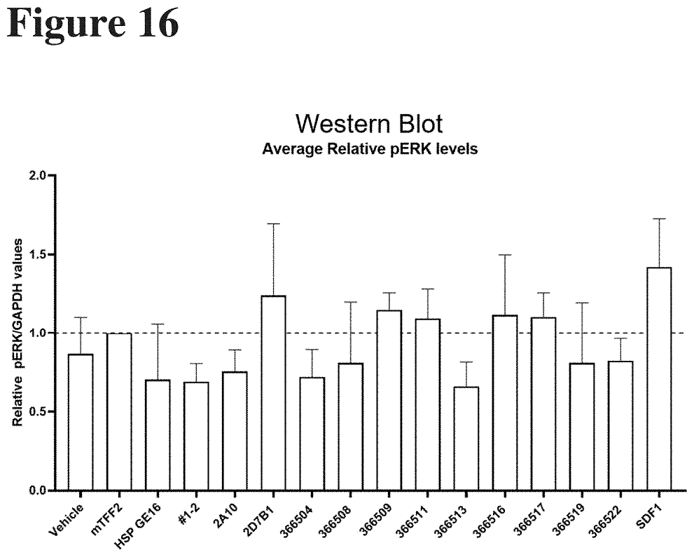

FIG. 16 shows the effects that thirteen anti-TFF2 antibodies from FIG. 14 had on TFF2 activity in Jurkat cells and demonstrates that several anti-TFF2 antibodies can inhibit TFF2 activity in Jurkat cells. A Western Blot TFF2 bioassay was performed for each anti-TFF2 antibody. Jurkat cells were grown in RPMI media with 10% FBS in a T-75 flask to confluency. Cells were counted, and 10⁷ cells were resuspended in in RPMI with no FBS and incubated overnight at 37° C., 5% CO₂. Serum starved cells were counted, and 2×10⁵ cells were added to sample tubes. Cells were treated with vehicle, TFF2, or positive control mouse SDF-1. Anti-TFF2 antibodies to be tested were added to the cells at 4 μg/ml, and samples were incubated at 37° C., 5% CO₂ for 15-30 min. Cells were lysed in RIPA with protease and phosphatase inhibitors, and samples were run on a 4-12% Bis-Tris gel in MOPS buffer. Gels were transferred to nitrocellulose membranes using the Trans-Blot Turbo transfer. After membrane transfer, blots were blocked for 1 hour in 5% BSA and probed with a rabbit anti-phospho ERK1/2 and GAPDH antibodies overnight at 4° C. in 5% BSA. Membranes were washed and appropriate secondary antibodies conjugated to HRP were incubated for 1 hour at RT before developing and imaging using a BioRad ChemiDoc system. Bands were quantified using Image Lab software for band intensity and normalized to GAPDH loading control blotted from on the same membrane. FIG. 16 shows the normalized relative pERK/GAPDH values from Western Blots demonstrating the treatment of Jurkat cells with the thirteen anti-TFF2 antibodies. The figure shows the results for treatment of Jurkat cells with a concentration of 4 μg/ml for each of the thirteen anti-TFF2 antibodies listed in FIG. 14 compared to treatment with a vehicle, TFF2, and a positive control (mouse SDF-1).

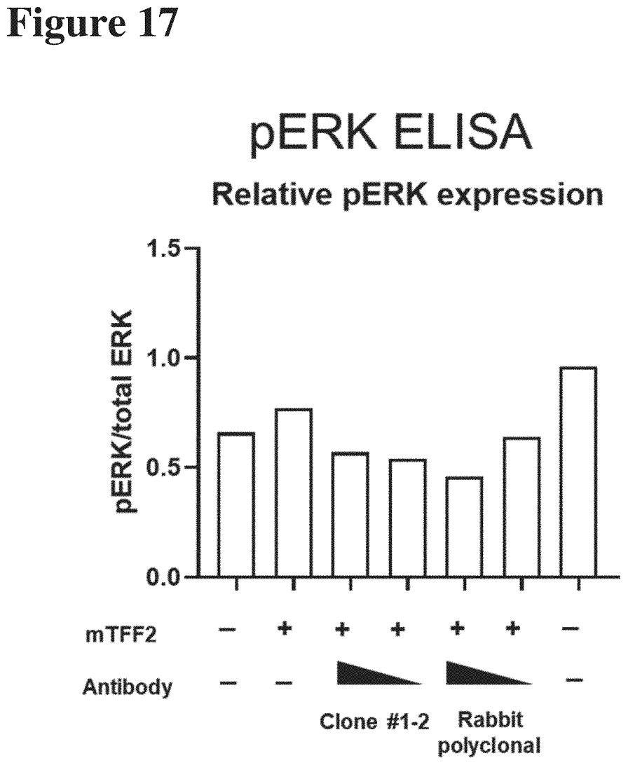

FIG. 17 shows that a specific commercially available monoclonal anti-hTFF2 antibody, Clone #1-2, neutralizes mouse TFF2 activity in Jurkat cells. Testing was performed using phospho-ERK1/2 ELISA. The TFF2 bioassay was performed, and the pERK ELISA was performed according to manufacturer's instructions (Thermo Fisher). Jurkat cells were grown in RPMI media with 10% FBS in a T-75 flask to confluency. Cells were counted, and 10⁷ cells were resuspended in in RPMI with no FBS and incubated overnight at 37° C., 5% CO₂. Serum starved cells were counted, and 2×10⁵ cells were added to sample tubes. Cells were treated with vehicle, TFF2, or positive control mouse SDF-1. Anti-TFF2 antibodies were added to the cells, and samples were incubated at 37° C., 5% CO₂ for 15-30 min. Cells were lysed with Cell Lysis Mix (5×) and shaken (˜300 rpm) at room temp for 10 minutes. Prepared sample lysate and positive and negative controls were added to the InstantOne ELISA™ assay wells. An antibody cocktail containing the detection and capture antibodies were added to each of the testing wells, and the microplate was then incubated for 1 hour at room temperature on a microplate shaker (˜300 rpm). After appropriate washing of the wells, detection reagent was added and incubated for 15 minutes with shaking at 300 rpm. After adding stop solution, the plate was read using a ClarioStar Plus plate reader set at 450 nm to measure the absorbance of the samples.

i. Human TFF2 Antibodies Improve Cognitive Performance In Vivo

To test the effects of TFF2 inhibition, 24-month-old aged C57Bl/6 mice were treated with a mouse anti-human TFF2 monoclonal antibody (n=15) or a control antibody (n=12) and analyzed for behavioral and gene expression readouts. The TFF2 antibody, Clone 366513 from R&D Systems, was raised against E. coli-derived recombinant human TFF2 (Glu24-Tyr129, provided as SEQ ID NO: 04) and purified from hybridoma supernatant. Clone 366513 is a commercially available sister clone to Clone 366508. The IgG2B isotype control antibody was purchased from Ichor Bio (Catalog #MPC-11). For both antibodies, mice were dosed at 1 mg/kg via intraperitoneal injection every 3 days for 4 weeks. Dosage amount and frequency were determined in a pharmacokinetic study. Prior to antibody treatment, mice were randomized into two groups such that there were no differences in average weight, cognitive performance in a spatial learning task (Y-Maze), or movement (measured by distance travelled and velocity).

The aged mice were tested in a contextual fear conditioning assay, which determines hippocampal-dependent cognition as measured by retention of a context-dependent fear memory. In this assay, mice were placed in a novel chamber and given an aversive stimulus of foot shocks (Training). One day later, mice were returned to the chamber, and the amount of time spent freezing during a 3-minute test period was recorded (Context Testing). A high percentage of time spent freezing during the test phase corresponds to an intact memory of the context. Mice were then sacrificed, and the hippocampus was dissected for qPCR analysis.

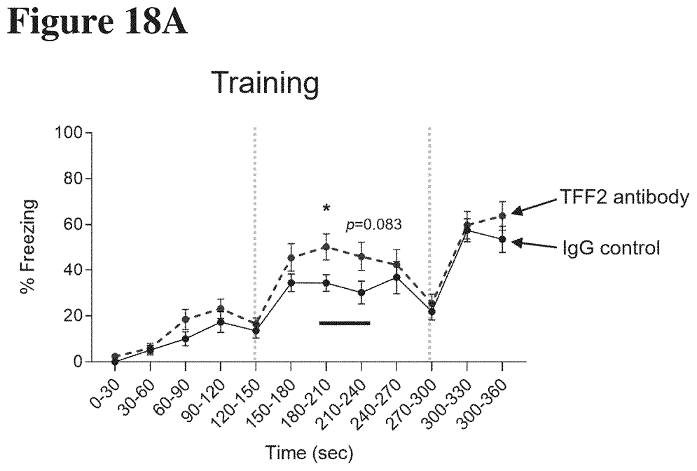

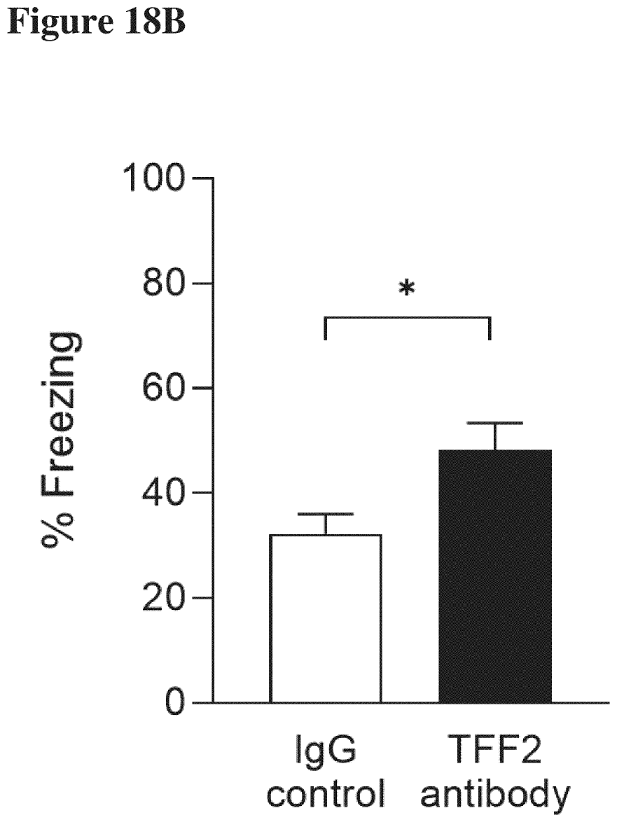

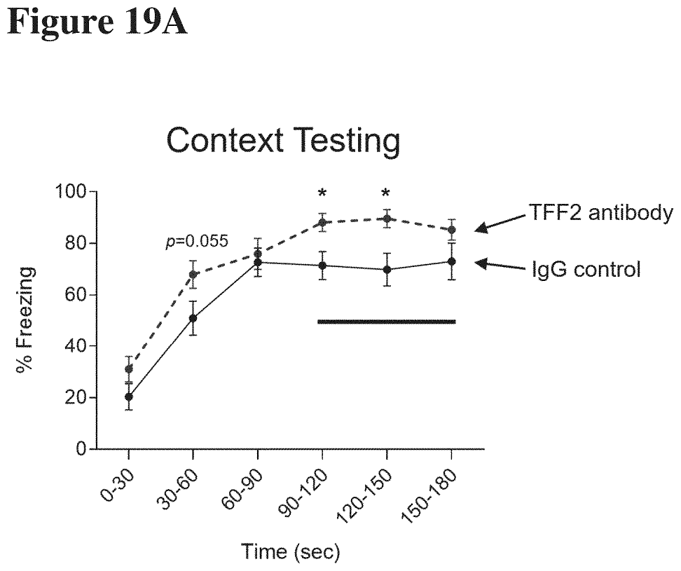

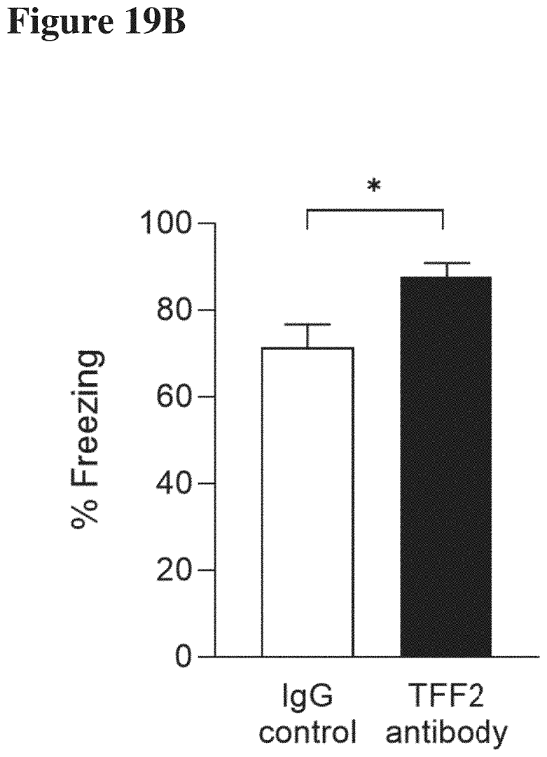

FIG. 18A shows that aged mice treated with human anti-TFF2 antibody froze more after foot shock during training. The mice spent a greater percentage of time freezing after foot shock compared to mice which were administered the control antibody. The Mann-Whitney test was used to determine significance for 30 second bins. * p=0.036. Vertical dotted lines correspond to the time when foot shock was administered. FIG. 18B shows that a significant increase in freezing can be detected for 1 minute after the first foot shock from FIG. 19A. (Mann-Whitney test, * p=0.032, n=15 TFF2 antibody, n=12 control antibody. All data are shown as mean±SEM).

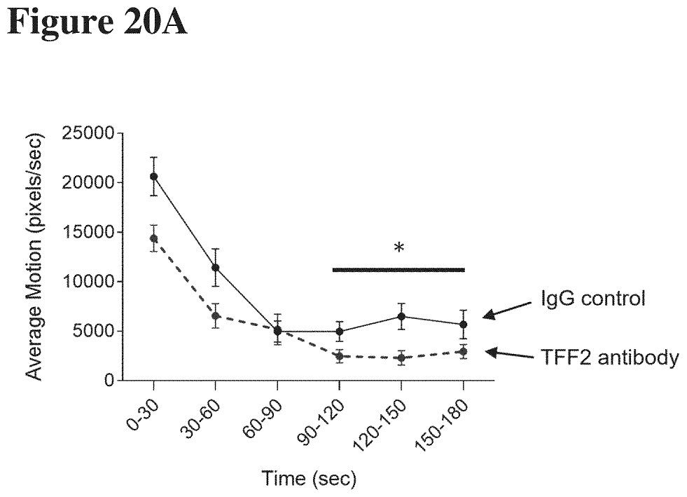

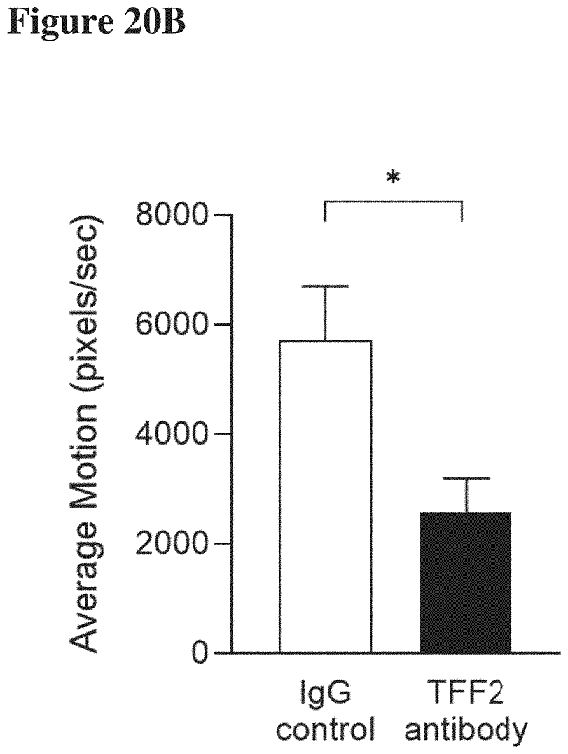

FIG. 19A shows that aged mice treated with human anti-TFF2 antibody retained memory better than mice treated with control antibody as determined by the contextual fear conditioning assay. TFF2 antibody-treated mice spent a greater percentage of time freezing compared to mice administered the control antibody during context testing. The Mann-Whitney test was used to determine significance for 30 second bins. * p<0.016. FIG. 19B shows that significant improvement in cognition occurs during the final half of the assay. (Mann-Whitney test, * p=0.025, n=15 TFF2 antibody, n=12 control antibody. All data are shown as mean±SEM). FIG. 20A shows that aged mice treated with human anti-TFF2 antibody moved less during contextual fear conditioning testing. A corresponding decrease in movement was observed in mice treated with the TFF2 antibody compared to controls. Two-way repeated measure ANOVA test was used to determine significance. p=0.025. FIG. 20B shows that mice treated with TFF2 antibody had less movement during the final half of the assay. (Mann-Whitney test, * p=0.025, n=15 TFF2 antibody, n=12 control antibody. All data are shown as mean±SEM).

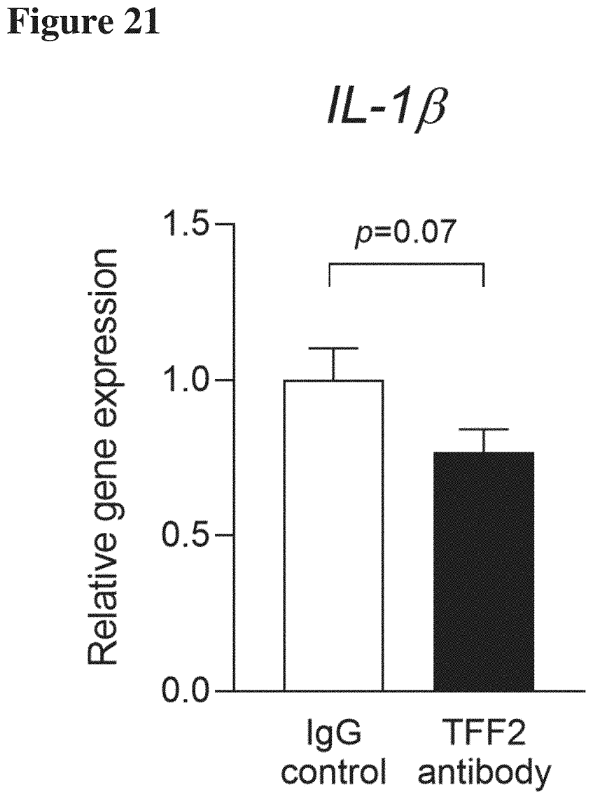

FIG. 21 shows that in the hippocampi of mice that underwent the contextual fear conditioning assay and treated with human anti-TFF2 antibody as described above, there was a downward trend in expression of the inflammatory marker, IL-1β compared to control antibody treated mice. This indicates that anti-TFF2 antibody treated mice trended towards decreased hippocampal inflammation, which accompanies aging and cognitive impairment. Expression was determined by qPCR. (Student's t-test, p=0.07, n=15 TFF2 antibody, n=12 control antibody. Data shown as mean±SEM).

1. A method of treating an adult mammal for an aging-associated impairment, the method comprising:
administering to the adult mammal a trefoil factor family member 2 (TFF2) level reducing agent in a manner sufficient to increase the cognitive performance of the mammal and treat the adult mammal for the aging-associated cognitive impairment.

2. The method according to claim 1, wherein the TFF2 level reducing agent is an antibody or a binding fragment thereof.

3. The method according to claim 2, wherein the antibody or binding fragment thereof was raised by inoculating a mammal with the protein of SEQ ID NO: 03

4. The method according to claim 2, wherein the antibody or binding fragment thereof was raised by inoculating a mammal with the protein of SEQ ID NO: 04.

5. The method according to claim 2, wherein the antibody is a monoclonal antibody.

6. The method according to claim 2, wherein the antibody or binding fragment is bound to a fixed substrate.

7. The method according to claim 1, wherein the TFF2 level reducing agent is a small molecule.

8. The method according to claim 1, wherein the TFF2 level reducing agent is a TFF2 expression modulatory agent.

9. The method according to claim 1, wherein the mammal is a primate.

10. The method according to claim 9, wherein the primate is a human.

11. The method according to claim 1, wherein the adult mammal is an elderly mammal.

12. The method according to claim 11, wherein the elderly mammal is a human that is 60 years or older.

13. The method according to claim 1, wherein the aging-associated impairment comprises a cognitive impairment.

14. The method according to claim 1, wherein the aging-associated impairment is mild cognitive impairment.

15. The method according to claim 13, wherein the cognitive impairment is associated with the group consisting of: Alzheimer's disease, Parkinson's disease, frontotemporal dementia, Huntington's disease, and amyotrophic lateral sclerosis.

16. The method according to claim 2, wherein the antibody binds to an antigen selected from the group consisting of SEQ ID NO: 02, SEQ ID NO: 04, SEQ ID NO: 06, SEQ ID NO: 08, SEQ ID NO: 10, and SEQ ID NO: 12.

17. The method according to claim 2, wherein the antibody binds to an antigen selected from the group consisting of SEQ ID NO: 02, SEQ ID NO: 04, SEQ ID NO: 06, SEQ ID NO: 08, SEQ ID NO: 10, and SEQ ID NO: 12.
